(19) United States
(12) Patent Application Publication
(10) Pub. No.: US 2025/0195576 A1
(43) Pub. Date: Jun. 19, 2025

(54) METHOD TO GENERATE MORE EFFICIENT CAR-T CELLS

(71) Applicants: INSTITUT NATIONAL DE LA SANTÉ ET DE LA RECHERCHE MÉDICALE, PARIS (FR); CENTRE NATIONAL DE LA RECHERCHE SCIENTIFIQUE, PARIS (FR); UNIVERSITE PARIS CITE, PARIS (FR)

(72) Inventors: Marianne MANGENEY, Paris (FR); Maude MARCHAIS, Paris (FR)

(73) Assignees: INSTITUT NATIONAL DE LA SANTÉ ET DE LA RECHERCHE MÉDICALE, PARIS (FR); CENTRE NATIONAL DE LA RECHERCHE SCIENTIFIQUE, PARIS (FR); UNIVERSITE PARIS CITE, PARIS (FR)

(21) Appl. No.: 19/066,980

(22) Filed: Feb. 28, 2025

Related U.S. Application Data
(63) Continuation-in-part of application No. PCT/EP2023/073799, filed on Aug. 30, 2023.

(30) Foreign Application Priority Data
Aug. 31, 2022 (EP) 22306289.4

(51) Int. Cl. A61K 35/17; A61K 40/11; A61K 40/31; A61P 35/00; C12N 5/00; C12N 5/0783

(52) U.S. Cl. CPC A61K 35/17; A61K 40/11; A61K 40/31; A61P 35/00; C12N 5/0018; C12N 5/0636; A61K 2239/38; C12N 2501/2307; C12N 2501/2315; C12N 2501/60; C12N 2510/00

(57) ABSTRACT

A method to obtain more effective CAR-T cells, which are FOXO1 inhibited. The inhibition of FOXO1 potentiates the ability to induce lysis of a target cell thanks to the increased expression of TNF-α and other inflammatory cytokines, induces spontaneous cell polarization comparable to that obtained by stimulation with chemokines and thus improves the motility of the cells, induces a sharp increase of memory T cells and allows to obtain more efficient CAR-T cells to treat solid tumors than classical CAR-T cells obtained with the known protocol. An 10 ex vivo method to obtain improved CAR-T cells including the steps of (i) cultivate T cells obtained from a subject with a FOXO1 inhibitor during a time of 2 to 10 days; (ii) transducing (or transforming) the T cells into CAR-T cells thanks to a known method.

### FIELD

The present invention relates to an ex vivo method to obtain improved CAR-T cells comprising the following steps i) cultivate T-cells obtained from a subject with a FOXO1 inhibitor during a time of 2 to 10 days; ii) transducing (or transforming) the T cells into CAR-T cells thanks to a known method.

### BACKGROUND

Chimeric Antigen Receptor T-cells (CAR-T cells) represent a very promising treatment in cancer. CARs are synthetic immune receptors that link antigen binding domains, commonly a single chain variable fragment (scFv), with T cell signaling domains, to endow T cells with non-MHC restricted specificity to defined cell surface antigens. Clinical trials demonstrated impressive activity of CD19 CAR-T cells against B cell malignancies, and the U.S. Food and Drug Administration and the European Medicines Agency recently approved CAR-T cells therapy for the treatment of patients with B cell precursor ALL, diffuse large B cell lymphoma and primary mediastinal B cell lymphoma (Schuster et al., 2019; Neelapu et al., 2017). Although this treatment gives remarkable results for some hematological cancers, 30% of patients relapse. In addition, the effectiveness of this treatment remains limited for solid tumors (Majzner & Mackall, 2019). Numerous studies have established that the ex vivo proliferation steps required for transduction of T cells by lentiviral particles induce a low engraftment potential of CAR-T cells, leading to a loss of antitumor activity (Klebanoff A C et al., 2017; Ghassemi et al., 2018). This deleterious effect could be mitigated by inhibiting signaling pathways mobilized during the expansion phases required to transduction and amplification of CAR-T cells (Bowers et al., 2017; Crompton et al., 2015; Klebanoff et al., 2017; Urak et al., 2017; van der Waart et al., 2014). An alternative approach would be to induce CAR expression in quiescent T cells in the absence of any stimulation in order to decrease the loss of antitumor activities associated with the activation and expansion of T cells ex vivo as shown in Ghassemi et al., 2022.

### SUMMARY

The inventors worked on the function of FOXO1 in T lymphocyte physiology for many years. They recently described, using a FOXO1 pharmacological inhibitor, that the acute block of this transcription factor induces a drastic metabolism modification of quiescent T cells, notably allowing their infection with lentiviruses (Roux et al., 2019). In this context, they recently used AS1842856 as a tool to investigate the control by FOXO1 of the expression of some still poorly studied targets of FOXO1 in human T lymphocytes. They observed during their experiments that AS1842856 treatment of human T cells purified from healthy donors caused, after a few days of culture and in the absence of any growth factor (cytokines, etc.), a substantial increase in their metabolic activity that correlates with acquisition of phenotypic and functional characteristics of activated/memory T cells. More specifically, they found that AS1842856 induces an increase in the production of granzyme B in CD8+ T cells but also in CD4+ T cells resulting in a potentiation of the cytotoxic activity of human CD8+ T primary lymphocytes.

Thanks to permissivity induced by FOXO1 inhibition, the inventors explored the possibility that a simple pharmacological treatment during ex vivo culture can generate CAR-T cells lacking the exhausted characteristics of classical CAR-T cells. They observed that inhibition of FOXO1 by AS1842856 pharmacological agent not only allows quiescent T cells infection but also the acquisition of phenotypic and functional characteristics leading to a strong increase of CAR-T cell antitumoral activity. Notably, they showed for the first time that the inhibition of FOXO1 potentiates the ability to induce lysis of a target cell thanks to the increased expression of TNF-α and other inflammatory cytokines, induces spontaneous cell polarization comparable to that obtained by stimulation with chemokines and thus improves the motility of the cells, induces a sharp increase of memory T cells and allows to obtain more efficient CAR-T cells to treat solid tumors than classical CAR-T cells obtained with the known protocol.

Thus, the present invention relates to an ex vivo method to obtain improved CAR-T cells comprising the following steps i) cultivate T-cells obtained from a subject with a FOXO1 inhibitor during a time of 2 to 10 days; ii) transducing (or transforming) the T cells into CAR-T cells thanks to a known method. Particularly, the invention is defined by its claims.

### BRIEF DESCRIPTION OF THE FIGURES

FIGS. 1A to 1C: FOXO1 inhibition allows increase number of cytotoxic T cells and increased their activity. Human primary T lymphocytes from healthy donor (PBT) were treated with AS1842856 (500 nM) or equivalent of DMSO for 7 days. (1A) Cells were surface labeled with anti-CD4 and anti-CD8, next fixed and permeabilized before labeled with antibodies against Granzyme B. The graphs present the values correspond to the mean±SEM obtained with three independent donors. Significance was assessed using a paired Student t-test. (1B) At the end of the culture, cells were stimulated by PMA and ionomycin during three hours. After CD4 and CD8 labelling, intracellular detection of TNF-α was performed using an antibody against TNF-α. The values correspond to the mean±SEM obtained with three independent donors. Significance was assessed using a paired Student t-test. (1C) CD8 T cells from vehicle or treated cells were purified and co-cultured with P815 target cells pre-coated with anti-CD3 mAb, percentage of lysis was evaluated by 51Cr release assay at different effector: target ratios. The values correspond to the mean±SEM obtained with three independent donors. Significance was assessed using 2-way Anova test.

FIGS. 2A and 2B: FOXO1 inhibition promotes T cells motility. PBT were treated with AS1842856 (500 nM) or equivalent of DMSO for 7 days. (2A) At the end of the culture, cells were stimulated or not with 100 ng/ml CCL19 for 8 min, fixed, stained for F-actin and shape deformation was analyzed using Image Stream. The graph presents the values correspond to the mean of results±SEM obtained with three independent donors. Significance was assessed using 2-way Anova test. (2B) Migration of T cells treated or not with AS1842856 in vibratome sections of viable Capan-2 tumors. The graphs present average speed and displacement±SEM of T cells treated or not with AS1842856 in Capan-2 tumor slices from three independent experiments. In each experiment a minimum of 100 cells were analyzed. Significance was assessed using a paired Student t-test.

FIGS. 3A and 3B: AS1842856 primes human T lymphocytes and induces the acquisition of a memory phenotype

PBT were treated with 500 nM of AS1842856 or vehicle only for 7 days. (3A) Memory subsets composition in vehicle or AS1842856 CD4 (upper panel) and CD8 (lower panel) cells. T_{N} are CD45RA+, CD27+, CD95−; T_{SCM} are CD45RA+, CD27+, CD95+; T_{CM} are CD45RA−, CD27+; T_{EM} are CD45RA−, CD27− and Teff are CD45RA+, CD27−. The graphs show the mean percentage±SEM of the different subsets in vehicle or AS1842856 conditions on CD4 (upper panel) and CD8 (lower panel) T cells obtained with five independent donors. Significance was assessed using 1-way Anova test. (3B) CTLA-4, PD-1 and TIGIT expressions on CD4 (upper panel) and CD8 (lower panel) T cells were measured by FACS. The graphs show the MFI±SEM obtained with five independent donors. Significance was assessed using a paired Student t-test.

FIG. 4: FOXO1 inhibition increase cell survival

PBT were treated with AS1842856 (500 nM) or vehicle only. Every seven days, some cells from the two conditions were collected and labeled with Propidium Iodide (PI). The graph shows the mean percentage±SEM of viable cells (meaning PI−) in vehicle or AS1842856 conditions considering results on 4 different donors. Significance was assessed using 2-way Anova test.

FIGS. 5A to 5C: CAR-T^{AS} cells are more potent to eradicate tumor as classical CAR-T cells

NSG mice were engrafted with Capan-2LUC tumors in right flank at day-7. At day 0 PBS, 2×10⁵ CAR-T or CAR-T^{AS} cells were intravenously injected. (5A) Bioluminescence imaging were performed at the indicated days post CAR-T cells injection by injected i.p. of luciferine. The graph shows the median±SEM of signal intensity of tumor. Statistical analyzes were obtained with 2way Anova and the p-value corresponds to the comparison with the PBS condition (5B) At day 40 days, the measure of tumor was performed by ultrasound scanning. (5C) At days 41, after euthanasia tumors were weighted. For (B-C), the graph presented the median±SEM obtained with 6 animals per group. Statistical analyzes were calculated with Student t-test.

FIGS. 6A to 6C: CAR-TAS cells generated from patients' cells show increased proliferative capacity. CAR-T cells and CAR-TAS cells were generated from patients as described above. (6A) A FACS analysis of memory subsets distribution. (6B) Mean percentage of the identified populations±SEM Tim3+ cells and LAG-3+ cells in patients' CAR-T cells and patients' CAR-TAS cells. (6C) Comparison of cell expansion of CAR-T or CAR-TAS cells generated from patients after TransAct stimulation alone. The graphs show the fold increase for each day from the number of cells at day 0 during 10 to 28 days. CAR-T cells are in black and CAR-TAS cells in red.

FIG. 7: In vivo tumor growth control required 10⁷ cells of CAR-T cells not treated with FOXO1 inhibitor. NSG mice were engrafted with Capan-2^{LUC} tumors in the right flank at day −7. At day 0 PBS, 2×10⁵, 5×10⁵ or 10⁷ CAR-T cells were intravenously injected. Bioluminescence imaging were performed at the indicated days post CAR-T cells injection by injected i.p of luciferine. The graph presents the mean±SEM of normalized intensities of individual.

FIG. 8: CAR-T^{AS} cells are more potent to eradicate tumors than classical CAR-T cells in an orthotopic model. NSG mice were intravenously injected with A549^{LUC} tumors in the tail vein at day-3. At day 0 PBS, 2×10⁵ CAR-T or CAR-T^{AS} cells were intravenously injected in the retro-orbital vein. Bioluminescence imaging was performed at the indicated days post CAR-T cell injection by injection i.p. of luciferine. The graph presents the median obtained with 6 animals per group.

### DETAILED DESCRIPTION

FOXO1 inhibitor could be a very valuable tool to be used in therapies requiring ex vivo protocol of antitumor T cells to obtain CAR-T cells. Notably, FOXO1 inhibitor can be very useful in a protocol to obtain CAR-T cells to suppress the step of activation and the step of expansion of the T cells which means without activator of the TCR of the cells like antibodies directed against CD3 and CD28 molecules. The inventors demonstrate that inhibition of FOXO1 not only allows infection of non-activated T cells, but also the acquisition of phenotypic and functional characteristics (notably less exhaustion markers) leading to a strong increase of CAR-T cells antitumor activity. They also show that T cell obtained with their protocol undergo a homeostatic proliferation after in vivo injection which induces the CAR expression. In other words, the new protocol of the inventors allows to obtain CAR-T cells quicker since no expansion and activation steps are needed where CAR expression is inducible by proliferation and allows to obtain more efficient CAR-T cells as explained above.

Thus, in a first aspect, the invention relates to an ex vivo method to obtain improved CAR-T cells comprising the following steps:
i) cultivate T-cells obtained from a subject with a FOXO1 inhibitor during a time of 2 to 10 days;
ii) transducing (or transforming) the T cells into CAR-T cells thanks to a known method.

Thus, in a particular aspect the invention relates to an ex vivo method to obtain improved CAR-T cells comprising the following steps:
i) providing T-cells from a subject;
ii) cultivate the T-cells with a FOXO1 inhibitor during a time of 2 to 10 days;
iii) transducing (or transforming) the T cells into CAR-T cells thanks to a known method.

After the FOXO1 inhibitor protocol, CAR-T cells obtained can be injected to a subject in need thereof.

Accordingly, in the methods of the invention, the transformation of the T cells in CAR-T cells is made without prior activation.

Accordingly, in the methods of the invention, the transformation of the T cells in CAR-T cells is made without prior ex vivo activation (e.g., activation using anti-CD3 and/or anti-CD28 antibodies) and expansion and therefore prevent activation-induced differentiation of T cells.

In particular embodiment, the T cells have been harvested from a subject prior to the methods of the invention. In particular embodiment, the T cells have been harvested from a subject prior to the methods of the invention and stored (in frozen state, i.e formulated in cryopreservation media).

In a particular embodiment, the method comprises another step of addition of IL-7 and/or IL-15 after the use of the inhibitor of FOXO1.

This also relates to an ex vivo method to obtain improved CAR-T cells comprising the following steps:
i) providing T-cells from a subject;
ii) cultivate the T cells in a medium with a FOXO1 inhibitor during a time of 2 to 10 days;
iii) add IL-7 and/or IL-15 to the medium;
iv) transducing (or transforming) the T cells into CAR-T cells thanks to a known method.

According to the methods explained above, the methods can be done in vitro.

In a particular embodiment, the IL-7 and/or IL-15 are administrated simultaneously with the FOXO1 inhibitor.

As used herein, the term T cells denotes for example CD3+ T cells, CD4+ T cells, CD8+ T cells, TILs T cells (Tumor-infiltrating lymphocytes T cells), NK T cells. These T cells can be isolated from peripheral blood lymphocytes (PBL) or peripheral blood mononuclear cells (PBMC) or from a biopsy when these cells are for example TILs.

In particular embodiment, T cells are non-activated T cells (i.e not activated via T cell receptor (TCR) or co-receptors such as CD3 and/or CD28). In particular embodiment, T cells are quiescent cells.

As used herein the term “quiescent T cells” has its general meaning in the art and refers to non-proliferating, non-dividing, or resting T cells in the G0 phase of the cell cycle. T cells may naturally be in a quiescent state.

In particular embodiment, the medium is medium suitable to the culture of T cell, namely optimized for T cell culture. Medium suitable for the culture of T cell are commercially available and include but are not limited to RPMI 1640 basal medium supplemented or not with 10% human serum, in particularly human AB serum.

According to the invention, the T cells can be in contact with the FOXO1 inhibitor during a time of 2, 3, 4, 5, 6, 7, 8, 9 or 10 days.

After the end of the protocol and the obtention of the CAR-T cells, these cells are injected to a subject in need thereof.

According to the invention, the concentration of the FOXO1 inhibitor is between 50 and 1000 nM. Particularly, the concentration is 50, 100, 200, 300, 400, 500, 600, 700, 800, 900 or 1000 nM. Particularly, the concentration is 500 nM.

According to the invention, the quantity of T cells collected in a subject in need thereof and used in the methods of the invention is between 10⁴ and 10⁹ cells per kg. Particularly, the concentration of T cells used in the protocol is 5×10⁶ cells.

In other words, the quantity of blood collected in a subject in need thereof to obtain the appropriate quantity of T cells is between 5 to 100 ml. Particularly, the quantity of blood collected is 5, 10, 20, 30 40 or 50 ml.

As used herein, “FOXO1” belongs to Forkhead Box class O transcription factors which are known to be key molecules to regulate and maintain cell quiescence in various cell types. In unstimulated cells, these transcription factors are in the nucleus, unphosphorylated and active, thereby maintaining the transcription of numerous genes. They act as key regulators to coordinate signals delivered by growth factors to molecular events leading to cell growth and cell division. FOXO1 corresponds to the most abundant FOXO molecule present in T cells (Entrez Gene ID number: 2308).

As used herein “FOXO1 inhibitor” denotes an inhibitor which induces a transition from quiescence G0 to the G1 phase of the cell cycle. For example, the effect is obtained by the AS1842856 compound by inhibiting binding of FOXO1 on the DNA. Without altering the phosphorylation state or expression of FOXO1, AS1842856 would keep the role of FOXO1 on chromatin remodelling. The use of the inhibitor of the present invention induces a stem cell memory phenotype (T_{SCM}), together with a high granzyme B expression and an increased tumor necrosis factor alpha secretion. After treatment of T cells by a FOXO1 inhibitor, the cells present an enhanced proliferative capacity, an improved cytotoxic potential, improved migratory properties and improved efficiency to eradicate tumors in vivo.

In view of the foregoing, the invention also relates to FOXO1 inhibited CAR-T cells (e.g. CAR-T^{AS} cells) presenting: an anti-tumor activity which is increased compared to CAR-T cells non-treated by FOXO1 inhibitor (previously named “classical CAR-T cells”); and a cell expansion which is increased compared to CAR-T cells non-treated by FOXO1 inhibitor. Additionally, said FOXO1 inhibited CAR-T cells further lack the exhausted characteristics of CAR-T cells non-treated by FOXO1 inhibitor.

As used herein “anti-tumor activity which is increased compared to CAR-T cells non-treated by FOXO1 inhibitor” refers to the fact that the anti-tumor activity of 2×10⁵ FOXO1 inhibited CAR-T cells leads to a tumor regression in vivo in a subcutaneous xenograft model of pancreatic Capan-2 cell line expressing luciferase tumor cells, injected in immunodeficient NSG mice, with tumor regression being quantitatively assessed by bioluminescence imaging, whereas 2×10⁵ CAR-T cells non-treated by FOXO1 inhibitor leads to non-controlled tumor in the same subcutaneous xenograft model. Interestingly, data from which said characteristic has been measured have also demonstrated that at least 50 times fewer of FOXO1-inhibited CAR-T cells than CAR-T cells non-treated by FOXO1 inhibitor achieve to the same tumor regression in vivo in a subcutaneous xenograft model of pancreatic Capan-2 cell line expressing luciferase tumor cells, injected in immunodeficient NSG mice, with tumor regression being quantitatively assessed by bioluminescence imaging.

As used herein “anti-tumor activity which is increased compared to CAR-T cells non-treated by FOXO1 inhibitor” may also refers to the fact that the anti-tumor activity of 2×10⁵ FOXO1 inhibited CAR-T cells leads after 28 days to the inhibition of at least twice of a tumor progression in vivo in an orthotopic model of A549 cell line expressing luciferase tumor cells, injected in immunodeficient NSG mice, with tumor progression being quantitatively assessed by bioluminescence imaging, as compared to 2×10⁵ CAR-T cells non-treated by FOXO1 inhibitor in the same orthotopic model.

As used herein “cell expansion which is increased compared to CAR-T cells non-treated by FOXO1 inhibitor” refers to the fact cell expansion of FOXO1 inhibited CAR-T cells is associated with a cell expansion at least 3-fold compared to CAR-T cells non-treated by FOXO1 inhibitor after 10 days of in vitro stimulation.

As used herein “lack the exhausted characteristics of CAR-T cells non-treated by FOXO1 inhibitor” refers to the fact that FOXO1 inhibited CAR-T cells are associated with a reduction in the percentage of positive FOXO1 inhibited CAR-T cells of at least 2-fold for Tim3 marker and a reduction in the percentage of positive FOXO1 inhibited CAR-T cells of at least 5-fold for LAG3 marker.

### CAR-T Cells

As used herein, the terms “Chimeric antigen receptors (CARs)” refer to artificial T cell receptors, chimeric T cell receptors, or chimeric immunoreceptors, and encompass engineered receptors that graft an artificial specificity onto a particular immune effector cell, i.e. the T cells of the invention.

A CAR typically comprises an ectodomain (extracellular domain) and an endodomain (cytoplasmic domain), joined by a transmembrane domain. The ectodomain, expressed on the surface of the cell, comprises an antigen binding domain or receptor domain and optionally a spacer (or hinge) region linking the antigen binding domain to the transmembrane domain. The transmembrane domain is typically a hydrophobic alpha helix that spans across the lipid bilayer of the cell membrane. The endodomain of the CAR is composed of an intracellular signaling module that induces the cell activation upon antigen binding. The endodomain may include several signaling domains, as explained infra.

### Antigen Binding Domain

The extracellular domain of the CAR comprises an antigen binding domain that specifically binds or recognizes a target antigen.

As used herein, “bind” or “binding” refer to peptides, polypeptides, proteins, fusion proteins and antibodies (including antibody fragments) that recognize and contact an antigen. Preferably, it refers to an antigen-antibody type interaction. By “specifically bind” it is meant that the antigen binding domain of the CAR recognizes a specific antigen but does not substantially recognize or bind other molecules in a given sample. The “specific binding” is dependent upon the presence of a particular structure (e.g., an antigenic determinant or epitope). As used herein, the term “specific binding” means the contact between an antigen binding domain of the CAR and an antigen with a binding affinity of at least 10-6 M. In certain aspects, the antigen binding domain of the CAR binds with affinities of at least about 10-7 M, and preferably 10-8 M, 10-9 M, 10-10 M. The binding affinity can be measured by any method available to the person skilled in the art, in particular by surface plasmon resonance (SPR).

In one embodiment, such antigen binding domain is an antibody, preferably a single chain antibody. Preferably, the antibody is a humanized antibody. Particularly, such antigen binding domain is an antibody fragment selected from fragment antigen binding (Fab) fragments, F(ab′)2 fragments, Fab′ fragments, Fv fragments, recombinant IgG (rlgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antibodies are single-chain antibody fragments comprising a variable heavy chain region and/or a variable light chain region, such as scFv. Particularly, such antigen binding domain is selected from a Fab and a scFv.

In embodiments wherein, the antigen targeting domain is a scFv, the scFv can be derived from the variable heavy chain (VH) and variable light chain (VL) regions of an antigen-specific mAb linked by a flexible linker. The scFv retains the same specificity and a similar affinity as the full antibody from which it is derived. The peptide linker connecting scFv VH and VL domains joins the carboxyl terminus of one variable region domain to the amino terminus of the other variable domain without compromising the fidelity of the VH-VL paring and antigen-binding sites. Peptide linkers can vary from 10 to 30 amino acids in length. In one embodiment, the scFv peptide linker is a Gly/Ser linker and comprises one or more repeats of these amino acids.

The extracellular domain of the CAR may comprise one or more antigen binding domain(s).

In a particular embodiment, the CAR specifically binds to a tumor-associated antigen (TAA). In particular, the CAR specifically binds to any TAA expressed at the surface of a tumor cell, particularly CD19, GD2, EGFR, CD20, CD22, CD33, CD138, CD52, CD30, ROR1, HER2, EpCAM, MUC-1, MUC5AC, BCMA, CD38, SLAMF7/CS1, CD123, IL-13Ra2, LeY, MUC16, PSMA, more preferably the TAA is CD19, CD20, CD22, CD33, CD138, BCMA, CD38, SLAMF7/CS1, IL-13Ra2, HER2 or EGFR.

In another particular embodiment the CAR targets an intracellular oncoprotein or an intracellular tumor-associated antigen in particular WT-1, NY-ESO-1, MAGE, PRAME, RAS, mesothelin, c-Met, CEA, CSPG-4, EBNA3C, CA-125 or GPA7. In particular, said intracellular oncoprotein or tumor-associated antigen are processed and expressed on the cell surface as peptides bound to histocompatibility (HLA) molecules.

The terms “tumor-associated antigen”, “TAA”, “tumor antigen” and “cancer cell antigen” are used interchangeably herein. In each case, the terms refer to peptides, proteins, glycoproteins or carbohydrates that are specifically or preferentially expressed by cancer cells.

As used herein, the term “antigen” has its general meaning in the art and generally refers to a substance or fragment thereof that is recognized and selectively bound by an antibody or by a T cell antigen receptor, resulting in induction of an immune response. Antigens according to the invention are typically, although not exclusively, peptides and proteins. Antigens may be natural or synthetic and generally induce an immune response that is specific for that antigen.

As used herein, the term “HLA-A2” has its general meaning in the art and refers to a HLA serotype within the HLA-A ‘A’ serotype group and is encoded by the HLA-A*02 allele group including the HLA-A*02:01, HLA-A*02:02, HLA-A*02:03, HLA-A*02:05, HLA-A*02:06, HLA-A*02:07 and HLA-A*02:11 gene products. HLA-A2 is very common in the Caucasian population (40-50%) and provides an ideal cellular target for the first portion because it will be suitable for use in a high proportion of combinations of HLA-A2+ donors and HLA-A2-recipients.

As used herein the term “antibody” and “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

As used herein the term “human antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a KD of about 10-7 M or less, such as about 10-8 M or less, such as about 10-9 M or less, about 10-10 M or less, or about 10-11 M or even less.

### Spacer or Hinge Domain

The CAR optionally comprises a spacer or hinge domain linking the antigen binding domain to the transmembrane domain.

In some embodiments, the CAR comprises a hinge sequence between the antigen binding domain and the transmembrane domain and/or between the transmembrane domain and the cytoplasmic domain. One ordinarily skilled in the art will appreciate that a hinge sequence is a short sequence of amino acids that facilitates flexibility.

In particular, the spacer or hinge domain linking the antigen binding domain to the transmembrane domain is designed to be sufficiently flexible to allow the antigen binding domain to orient in a manner that allows antigen recognition.

The hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge domain includes at least a portion of an IgG1, an IgG2, an IgG3, an IgG4, an IgE, an IgM, or an IgA immunoglobulin Fc region that falls within its CH2 and CH3 domains.

Exemplary hinges include, but are not limited to, a CD8a hinge, a CD28 hinge, IgG1/IgG4 (hinge-Fc part) sequences, IgG4 hinge alone, IgG4 hinge linked to CH2 and CH3 domains, or IgG4 hinge linked to the CH3 domain, those described in Hudecek et al. (2013) Clin. Cancer Res., 19:3153, international patent application publication number WO2014031687, U.S. Pat. No. 8,822,647 or published app. No. US2014/0271635. As hinge domain, the invention relates to all or a part of residues 118 to 178 of CD8a (GenBank Accession No. NP_001759.3), residues 135 to 195 of CD8 (GenBank Accession No. AAA35664), residues 315 to 396 of CD4 (GenBank Accession No. NP_000607.1), or residues 137 to 152 of CD28 (GenBank Accession No. NP_006130.1) can be used. Also, as the spacer domain, a part of a constant region of an antibody H chain or L chain (CHI region or CL region) can be used. Further, the spacer domain may be an artificially synthesized sequence.

In some embodiments, for example, the hinge sequence is derived from a CD8 alpha molecule or a CD28 molecule.

### Transmembrane Domain

The transmembrane domain of the CAR functions to anchor the receptor on the cell surface. The choice of the transmembrane domain may depend on the neighboring spacer and intracellular sequences.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD134, CD137, CD154, ICOS/CD278, GITR/CD357, NKG2D, and DAP molecules. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. A transmembrane domain is thermodynamically stable in a membrane. It may be a single alpha helix, a transmembrane beta barrel, a beta-helix of gramicidin A, or any other structure.

Optionally, a short oligo- or polypeptide linker, preferably between 2 and 10 amino acids in length may form the linkage between the transmembrane domain and the intracellular signaling domain(s) of the CAR. A glycine-serine doublet may provide a suitable linker.

### Intracellular Domain

The terms “intracellular domain”, “cytoplasmic domain” and “intracellular signaling domain” are used interchangeably herein. The role of the intracellular domain of the CAR is to produce an activation signal to the T cell as soon as the extracellular domain has recognized the antigen.

Examples of intracellular domain sequences that are of particular use in the invention include those derived from an intracellular signaling domain of a lymphocyte receptor chain, a TCR/CD3 complex protein, an Fc receptor subunit, an IL-2 receptor subunit, CD3ζ, FcRγ, FcRβ, CD3Y, CD38, CD38, CD5, CD22, CD79a, CD79b, CD66d, CD278 (ICOS), FcsRI, DAP10, and DAP12. It is particularly preferred that the intracellular domain in the CAR comprises a cytoplasmic signaling sequence derived from CD35.

The intracellular domain of the CAR can be designed to comprise a signaling domain (such as the CD3ζ signaling domain) by itself or combined with costimulatory domain(s). A costimulatory molecule can be defined as a cell surface molecule that is required for an efficient response of lymphocytes to an antigen. Examples of such molecules include CD27, CD28, 4-1BB (CD137), OX40 (CD134), CD30, CD40, CD244 (2B4), ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. The intracellular signaling portion of the above recited co-stimulatory domains can be used alone or in combination with other co-stimulatory domains. In particular, the CAR can comprise any combination of two or more co-stimulatory domains from the group consisting of CD27, CD28, 4-1BB (CD137), OX40 (CD134), CD30, CD40, CD244 (2B4), ICOS, LFA-1, CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

Thus, for example, the CAR can be designed to comprise a signaling domain such as the CD32 signaling domain and two co-stimulatory signaling domains selected from CD28 and CD40, CD28 and 4-1BB (CD137), CD28 and OX40 (CD134), and CD28 and LFA-1.

“First-generation CARs” contain a single signaling domain. CARs containing a signaling domain together with one additional costimulatory domain are termed “second generation” while those containing a signaling domain together with two additional costimulatory domains are listed as “third generation”. For example, first-generation CARs contain solely the CD32 chain as a single signaling domain. Second- and third-generation CARs consist of one or two additional costimulatory signaling domains, respectively, such as CD28, CD27, OX-40 (CD134) and 4-1BB (CD137). For example, second-generation CAR may contain CD3ζ and CD28 signaling domains, while third-generation CAR may contain CD3ζ, CD28 and either OX40 (CD134) or 4-1BB (CD137).

The CAR of the invention may be a first generation, a second generation, or a third generation CAR as described hereabove. Preferably, the CAR-T cells is a second or third generation CAR.

“TRUCKs” represent the recently developed ‘fourth-generation’ CARs. TRUCKs (T cells redirected for universal cytokine killing) are CAR-redirected T cells used as vehicles to produce and release a transgenic product that accumulates in the targeted tissue. The product, for example a pro-inflammatory cytokine, may be constitutively produced or induced once the T cell is activated by the CAR. Other substances such as enzymes or immunomodulatory molecules may be produced in the same way and deposited by CAR-redirected T cells in the targeted lesion. This strategy involves two separate transgenes expressing for example (i) the CAR-T cells and (ii) a cell activation responsive promoter linked to a cytokine such as IL-12. Consequently, immune stimulatory cytokine such as IL-12 is secreted upon CAR engagement.

In a particular embodiment, the CAR-T cells is a CAR-T cells of fourth generation as defined above.

### Methods to Obtain a CAR-T Cells

Methods and protocols to obtain CAR-T cells are well known in the art. To obtain CAR-T cells from T cells, transfection, transposon system like the sleeping beauty method or infection thanks to a lentivirus can be used (see for example Martinez Marina et al., 2019).

Methods using lentivirus able to transduce T cells to obtain CAR-T cells are well known. For example, and as shown in the present application, a MOI2 lentivirus stock can be used. Protocols used to obtain CAR-T cells are well known in the art (see for example Okuma Atsushi, 2021. Generation of CAR-T Cells by Lentiviral Transduction).

Another method to obtain CAR-T cells from T cells is call sleeping beauty using DNA transposons to transfect the cells (see for example Izsvák et al. 2010).

According to the invention, the CAR-T cells can be CAR-T cells from the first, the second, the third or the fourth generation.

According to the invention, in the methods and protocols used to obtain CAR-T cell, none activation step (such as activation using anti-CD3 and/or anti-CD28 antibodies) is made prior transformation of T cells.

In particular embodiment, a polynucleotide encoding for the CAR is introduced (i.e via transfection or transduction) into the T cell to obtain CAR-T cells.

As used herein, the term “transformation”, “transfection” or “transduction” means the introduction of a “foreign” (i.e. extrinsic or extracellular) gene, DNA or RNA sequence to T cell, so that the T cell will express the introduced gene or sequence to produce a desired substance, i.e a CAR coded by the introduced gene or sequence. The T cell that receives and expresses introduced DNA or RNA bas been “transformed”.

Thus, in particular embodiment, the invention relates to an ex vivo method to obtain improved CAR-T cells comprising the following steps:
i) cultivate T-cells obtained from a subject in a medium with a FOXO1 inhibitor during a time of 2 to 10 days; and
ii) introduced into the T cells a polynucleotide encoding for the CAR to obtain CAR-T cells.

In a particular embodiment, the method comprises another step of addition of IL-7 and/or IL-15 in the medium after the use of the inhibitor of FOXO1.

It is contemplated that the polynucleotide encoding for the CAR can be introduced into the T cell as naked nucleic acid (DNA or RNA) or in a suitable vector.

Naked DNA generally refers to the DNA contained in a plasmid expression vector in proper orientation for expression. Physical methods for introducing a polynucleotide construct into T cell include particle bombardment, nucleofection, colloidal dispersion systems, such as macromolecule complexes, nanocapsules, microspheres, beads, and lipid-based systems including oil-in-water emulsions, micelles, mixed micelles, and liposomes and the like.

In some embodiments, the polynucleotide encoding for the CAR is introduced into the T cell by a viral vector that is an adeno-associated virus (AAV), a retrovirus, lentivirus, bovine papilloma virus, an adenovirus vector, a vaccinia virus, a polyoma virus, or an infective virus. In some embodiments, the vector is a retroviral. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, the polynucleotide of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign polynucleotide, for selection and for transfer of the polynucleotide into a host cell. The gag, pol and env genes of the vectors of interest also are known in the art. Thus, the relevant genes are cloned into the selected vector and then used to transform the target cell of interest. This describes a first vector that can provide a polynucleotide encoding a viral gag and a pol gene and another vector that can provide a polynucleotide encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. More preferably, the env is vesicular stomatitis virus (VSV-G). In some embodiments, the vector is a lentivirus.

Typically, the suitable vector of the present invention includes “control sequences'”, which refers collectively to promoter sequences, polyadenylation signals, transcription termination sequences, upstream regulatory domains, origins of replication, enhancers, and the like, which collectively provide for the replication, transcription and translation of a coding sequence in a recipient cell. Not all of these control sequences need always be present so long as the selected coding sequence is capable of being replicated, transcribed and translated in the T cell. Another polynucleotide sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. The polynucleotides encoding for the CAR of the present invention may be operably linked to inducible promoters or retroviral long terminal repeats (LTRs), cytomegalovirus (CMV) promoter, murine stem cell virus (MSCV) U3 promoter, phosphoglycerate kinase (PGK) promoter, β-actin promoter, ubiquitin promoter, and a simian virus 40 (SV40)/CD43 composite promoter; elongation factor (EF)-1α promoter; myeloproliferative sarcoma virus enhancer, negative control region deleted, d1587rev primer-binding site substituted (MND) promoter; or the spleen focus-forming virus (SFFV) promoter. The new method of the inventions allows to obtain CAR-T cells where CAR expression is inducible by proliferation. Thus, preferably, the polynucleotides encoding for the CAR of the present invention is not operably linked to an inducible promoters.

In some embodiments, the sequence of the polynucleotides encoding the CAR is codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. The phrase “polynucleotide encoding a CAR” may also include introns to the extent that the nucleotide sequence encoding the protein may in some version contain an intron(s).

In particular embodiment, the polynucleotide encoding for the CAR is introduced into T cell by a DNA transposons (“sleeping beauty transposon system”)

As used herein, the term “DNA transposon” has its general meaning in the art and refers to DNA transfer vehicles that are capable of efficient genomic insertion. DNA transposons translocate from one DNA site to another in a simple, cut-and-paste manner. The Sleeping Beauty transposon system is composed of a Sleeping Beauty (SB) transposase and a transposon designed to insert specific sequences of DNA into genomes of vertebrate animals, as disclosed in Izsvák et al. 2010.

### FOXO1 Inhibitors

In a particular embodiment the FOXO1 inhibitor is the AS1842856.

The term “AS1842856” refers to cell-permeable inhibitor that blocks the transcription activity of FOXO1 and which is specific to FOXO1.

In another embodiment the FOXO1 inhibitor can be the tanzawaic acid D, the hymenidin, the cribrostatin 6, the barbamide and the compound 10 (see Sun Yingjia et al., 2016 and Lee et al. 2021).

In one embodiment, the inhibitor according to the invention may be a low molecular weight compound, e.g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Particular small organic molecules range in size up to about 10000 Da, more particularly up to 5000 Da, more particularly up to 2000 Da and most particularly up to about 1000 Da.

The present invention provides for an isolated single domain antibody, wherein said antibody inhibit FOXO1.

As used herein the term “single domain antibody” has its general meaning in the art and refers to the single heavy chain variable domain of antibodies of the type that can be found in Camelid mammals which are naturally devoid of light chains. Such single domain antibody is also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21 (11): 484-490; and WO 06/030220, WO 06/003388. The nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus yet another consequence of small size is that a nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in nanobodies being extremely thermostable, stable to extreme pH and to proteolytic digestion, and poorly antigenic. Another consequence is that nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FRI-CDR1-FR2-CDR2-FR3-CDR3-FR4 in which FRI to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system aminoacid numbering (http://imgt.cines.fr/).

Camel Ig can be modified by genetic engineering to yield a small protein having high affinity for a target, resulting in a low molecular weight antibody-derived protein known as a “nanobody” or “VHH”. See U.S. Pat. No. 5,759,808 issued Jun. 2, 1998; see also Stijlemans, B. et al., 2004 J Biol Chem 279:1256-1261; Dumoulin, M. et al., 2003 Nature 424:783-788; Pleschberger, M. et al. 2003 Bioconjugate Chem 14:440-448; Cortez-Retamozo, V. et al. 2002 Int J Cancer 89:456-62; and Lauwereys, M. et al. 1998 EMBO J 17:3512-3520. Engineered libraries of camelid antibodies and antibody fragments are commercially available, for example, from Ablynx, Ghent, Belgium. In certain embodiments herein, the camelid antibody or nanobody is naturally produced in the camelid animal, i.e., is produced by the camelid following immunization with [antigen] or a peptide fragment thereof, using techniques described herein for other antibodies. Alternatively, the [antigen]-binding camelid nanobody is engineered, i.e., produced by selection for example from a library of phage displaying appropriately mutagenized camelid nanobody proteins using panning procedures with FOXO1 as a target.

In some embodiments, the single domain antibody is a “humanized” single domain antibody.

As used herein the term “humanized” refers to a single domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favourable properties of single domain antibodies of the invention. The one skilled in the art is able to determine and select suitable humanizing substitutions or suitable combinations of humanizing substitutions. For example, the single domain antibodies of the invention may be suitably humanized at any framework residue that the single domain antibodies remain soluble and do not significantly loss their affinity for FOXO1.

In another embodiment, the FOXO1 inhibitor according to the invention is an inhibitor of foxo1 gene expression.

Small inhibitory RNAs (siRNAs) can also function as inhibitors of foxo1 expression for use in the present invention. DHODH or Chk1 gene expression can be reduced by contacting a subject or cell with a small double stranded RNA (dsRNA), or a vector or construct causing the production of a small double stranded RNA, such that foxo1 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of foxo1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of DHODH or CHk1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of foxo1 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and particularly cells expressing FOXO1. Particularly, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a particular type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Particular viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes. For example, a specific expression in Muller glial cells may be obtained through the promoter of the glutamine synthetase gene is suitable. The promoter can also be, e.g., a viral promoter, such as CMV promoter or any synthetic promoters.

In a particular embodiment, an endonuclease can be used to abolish the expression of the gene, transcript or protein variants of FOXO1.

Indeed, as an alternative to more conventional approaches, such as cDNA overexpression or downregulation by RNA interference, new technologies provide the means to manipulate the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone non-homologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339:823-826), bacteria (Fabre et al., 2014, PLOS Negl. Trop. Dis., Vol. 8: e2671.), zebrafish (Hwang et al., 2013, PLOS One, Vol. 8: e68708.), C. elegans (Hai et al., 2014 Cell Res. doi: 10.1038/cr.2014.11.), bacteria (Fabre et al., 2014, PLOS Negl. Trop. Dis., Vol. 8: e2671.), plants (Mali et al., 2013, Science, Vol. 339:823-826), Xenopus tropicalis (Guo et al., 2014, Development, Vol. 141:707-714.), yeast (DiCarlo et al., 2013, Nucleic Acids Res., Vol. 41:4336-4343.), Drosophila (Gratz et al., 2014 Genetics, doi: 10.1534/genetics.113.160713), monkeys (Niu et al., 2014, Cell, Vol. 156:836-843.), rabbits (Yang et al., 2014, J. Mol. Cell Biol., Vol. 6:97-99.), pigs (Hai et al., 2014, Cell Res. doi: 10.1038/cr.2014.11.), rats (Ma et al., 2014, Cell Res., Vol. 24:122-125.) and mice (Mashiko et al., 2014, Dev. Growth Differ. Vol. 56:122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein, the term “treatment” or “treat” refers to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness (e.g., the pattern of dosing used during therapy). A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “subject” denotes a mammal, such as a rodent, a feline, a canine, and a primate. Particularly, the subject according to the invention is a human.

### Use of the CAR-T Cells of the Invention

FOXO1 inhibited CAR-T cells, in particular CAR-T cells obtained by the methods of the invention, preferably CAR-T^{AS} cells, can be used to improve the immune response and thus can be used to treat diseases where the boost of the immune system is sought like cancer and infectious diseases.

Thus, a second aspect of the invention relates to FOXO1 inhibited CAR-T cells or CAR-T cells obtained (or produced) by a method of the invention to improve the immune response.

Particularly, the invention relates to FOXO1 inhibited CAR-T cells or CAR-T cells obtained by a method of the invention for use in the treatment of cancer or an infectious disease.

The invention also relates to a method to improve the immune system using FOXO1 inhibited CAR-T cells or CAR-T cells obtained by a method of the invention.

The invention also relates to a method to treat a cancer or an infectious disease using FOXO1 inhibited CAR-T cells or CAR-T cells obtained by a method of the invention.

Thus in other words, the invention relates to a method treat a cancer or an infectious disease in a subject in need thereof comprising administering to the subject a therapeutically effective amount of a FOXO1 inhibited CAR-T cells or CAR-T cells obtained by a method of the invention.

In particular embodiment, the CAR-T improves the subject's immune system.

More, the population of FOXO1 inhibited CAR-T cells or CAR-T cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. Adoptive immunotherapy of cancer refers to a therapeutic approach in which immune cells with an antitumor activity are administered to a tumor-bearing host, with the aim that the cells mediate either directly or indirectly, the regression of an established tumor. Transfusion of lymphocytes, particularly T lymphocytes, falls into this category.

According to the invention, the cancer may be a liquid or a solid cancer.

In one embodiment, the cancer may be a cancer selected from the group consisting in adrenal cortical cancer, anal cancer, bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma,), vaginal cancer, vulvar cancer, and uterine cancer (e.g. uterine leiomyosarcoma).

According to the invention, the infectious diseases can be due to a pathogen like a virus, bacterium, protozoan, prion, viroid, or fungus.

According to the invention, the bacterium can be selected from the group consisting of: Streptococcus pneumoniae; Staphylococcus aureus; Haemophilus influenza, Myoplasma species, Moraxella catarrhalis, Escherichia, e.g., E. coli, Enterobacter, Erwinia, Klebsiella, Proteus, Salmonella, e.g., Salmonella enterica serovar, Typhimurium, Serratia, e.g., Serratia marcescans, and Shigella, as well as Bacilli such as B. subtilis and B. licheniformis, Pseudomonas such as P. aeruginosa, Campylobacter, Mycobacterium tuberculosis, and Streptomyce.

According to the invention, the fungus can be selected from the group consisting of: aspergillus, Candida albicans and Cryptococcus neoformans.

More particularly, the infectious disease is induced by a respiratory virus.

Particularly, the respiratory virus can be Influenza virus, such as the Influenza A virus (IAV) or the Influenza B virus (IAB), adenovirus, metapneumovirus, cytomegalovirus, parainfluenza virus (e.g., hPIV-1, hPIV-2, hPIV-3, hPIV-4), the human rhinovirus (HRV), the Human respiratory syncytial virus (HRSV) or a coronavirus.

As used herein, the term “coronavirus” has its general meaning in the art and refers to any member of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. In particular, the coronavirus particle comprises at least the four canonical structural proteins E (envelope protein), M (membrane protein), N (nucleocapsid protein), and S (spike protein). The S protein is cleaved into 3 chains: Spike protein S1, Spike protein S2 and Spike protein S2′. Production of the replicase proteins is initiated by the translation of ORF1a and ORF1ab via a −1 ribosomal frame-shifting mechanism. This mechanism produces two large viral polyproteins, pp1a and pp1ab, that are further processed by two virally encoded cysteine proteases, the papain-like protease (PLpro) and a 3C-like protease (3CLpro), which is sometimes referred to as main protease (Mpro). Coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), Middle East respiratory syndrome-related coronavirus (MERS-Cov) and SARS-coronavirus (Center for Disease Control), in particular SARS-Cov1 and SARS-Cov2.

According to the invention, the coronavirus can be a MERS-COV, SARS-COV, SARS-CoV-2 or any new future family members.

Thus, particularly, the invention also relates to CAR-T cells obtained by the methods of the invention for use in the treatment of infectious disease induced by a pathogen as described above in a subject in need thereof.

### Therapeutic Composition

In a Third aspect, the invention relates to a therapeutic composition comprising FOXO1 inhibited CAR-T cells or CAR-T cells obtained by the method of the invention to improve the immune response.

In another embodiment, the invention relates to a therapeutic composition comprising FOXO1 inhibited CAR-T cells or CAR-T cells obtained by the method of the invention for use in the treatment of cancer and infectious disease.

According to the invention, the CAR-T cells are administrated in a therapeutically effective amount.

Any therapeutic agent of the invention may be combined with pharmaceutically acceptable excipients, and optionally sustained-release matrices, such as biodegradable polymers, to form therapeutic compositions.

As used herein, the term “therapeutically effective amount” or “effective amount” refers to an amount effective, at dosages and for periods of time necessary, to achieve a desired therapeutic result. A therapeutically effective amount of CAR-T cells of the present invention may vary according to factors such as the disease state, age, sex, and weight of the individual, and the ability of the CAR-T cells of the present invention to elicit a desired response in the individual. A therapeutically effective amount is also one in which any toxic or detrimental effects of the CAR-T cells of the present invention are outweighed by the therapeutically beneficial effects. The efficient dosages and dosage regimens for the combination of the CAR-T cells of the present invention depend on the disease or condition to be treated and may be determined by the persons skilled in the art. A physician having ordinary skill in the art may readily determine and prescribe the effective amount of the pharmaceutical composition required. For example, the physician could start doses of the oligomers of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of the CAR-T cells of the invention may, for example, be evaluated in an animal model system predictive of efficacy to treat cancer or infectious disease. Alternatively, this property of a composition may be evaluated by examining the ability of the compound to induce cytotoxicity by in vitro assays known to the skilled practitioner. A therapeutically effective amount of a therapeutic compound may decrease latent reservoirs, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of the CAR-T cells of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of the CAR-T cells of the present invention is 0.02-100 mg/kg, such as about 0.02-30 mg/kg, such as about 0.05-10 mg/kg or 0.1-3 mg/kg, for example about 0.5-2 mg/kg.

In other words, the quantity of CAR-T cells administered to a subject in need thereof is between 10⁴ to 10⁹ cells per kg. Particularly, the quantity of cells injected is 10⁶ or 10⁷ cells per kg. Particularly the unit to use the CAR-T cells of the invention will be most advantageously a number of cells per kg (as shown above).

Administration may be intravenous, intramuscular, intraperitoneal, intratumoral or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans. If desired, an effective daily dose of a pharmaceutical composition may be administered as two, three, four, five, six or more sub-doses administered separately at appropriate intervals throughout the day, optionally, in unit dosage forms. In some embodiments, the oligomers of the present invention are administered by slow continuous infusion over a long period, such as more than 24 hours, in order to minimize any unwanted side effects. An effective dose of the CAR-T cells of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of the CAR-T cells of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In other words, the quantity of CAR-T cells administered to a subject in need thereof is between 10⁴ to 10⁹ cells per kg. Particularly, the quantity of cells injected is 10⁶ or 10⁷ cells per kg. The CAR-T cells of the invention can be administrated is 1, 2, 3, 4 or 5 times to the subject in need thereof.

The CAR-T cells of the invention may be used alone or in combination with any suitable agent.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Pharmaceutical compositions of the present invention may comprise a further therapeutic active agent. The present invention also relates to a kit comprising an inhibitor according to the invention and a further therapeutic active agent.

For example, anti-cancer agents may be added to the pharmaceutical composition as described below.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The form of the pharmaceutical compositions, the route of administration, the dosage and the regimen naturally depend upon the condition to be treated, the severity of the illness, the age, weight, and sex of the subject, etc.

The pharmaceutical compositions of the invention can be formulated for a topical, oral, intranasal, parenteral, intraocular, intravenous, intramuscular or subcutaneous administration and the like.

Particularly, the pharmaceutical compositions contain vehicles which are pharmaceutically acceptable for a formulation capable of being injected. These may be in isotonic, sterile, saline solutions (monosodium or disodium phosphate, sodium, potassium, calcium or magnesium chloride and the like or mixtures of such salts), or dry, especially freeze-dried compositions. In particular, these may be in organic solvent such as DMSO, ethanol which upon addition, depending on the case, of sterilized water or physiological saline permit the constitution of injectable solutions.

In addition, other pharmaceutically acceptable forms include, e.g. tablets or other solids for oral administration; time release capsules; and any other form currently can be used.

In each of the embodiments of the treatment methods described herein, the CAR-T cells of the invention are delivered in a manner consistent with conventional methodologies associated with management of the disease or disorder for which treatment is sought. In accordance with the disclosure herein, an effective amount of the CAR-T cells of the invention administered to a subject in need of such treatment for a time and under conditions sufficient to prevent or treat the disease or disorder.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention will be further illustrated by the figures and following examples. However, these examples and figures should not be interpreted in any way as limiting the scope of the present invention.

### Example

### Material & Methods

Study approval. Human studies were carried out according to French law on biomedical research and to principles outlined in 1975 Helsinki Declaration and its modification. Institutional review board approval was obtained (CPP Ile de France II, #00001072, Aug. 27, 2012). Animal studies were approved by the animal experimentation ethics committee of Paris Descartes University (CEEA 34, 17-039) and by the French ministry of research (APAFIS #19762).

Cells. T lymphocytes were purified from the blood of healthy donors from the Etablissement Français du Sang (EFS, Paris, France) by Ficoll density gradient centrifugation followed by negative selection with EasySep™ Human T Cell Isolation Kit (Stem Cell, #17951) and cultured in RPMI 1640 GlutaMAX (Gibco, cat #61870-010) medium supplemented with 10% Human AB serum (Biowest, #S4190-100), penicillin and streptomycin (50U/ml and 50 μg/m respectively, penicillin-streptomycin from Thermo Fisher Scientific; cat #15140122) and 1 mM of sodium pyruvate (Gibco, cat #11360-039). TAS cells were obtained by 7 days treatment with 500 nM of AS1842856 (EMD Millipore, #344355). As AS1842856 was dissolved in DMSO, untreated cells were obtained by 7 days culture with the DMSO volume corresponding to AS1842856 dilution. P815 (ATCC®TIB-64), HEK293T (ATCC®CRL-11268), Capan-2 (ATCC® HTB-80) and A549 (ATCCCCL-185, kindly provided by Frederic Pendino, Institut Cochin, France) cells lines were maintained in culture in complete DMEM GlutaMAX (Gibco, cat #31966-021) containing 10% FBS, penicillin and streptomycin (50U/ml and 50 μg/m respectively). MT4R5 cell line (Amara et al. 2003 is maintained in culture in complete RPMI 1640 GlutaMAX containing 10% FBS, penicillin and streptomycin (50U/ml and 50 μg/m respectively) and 1 mM of sodium pyruvate. Each cell line was thawed from a lab frozen stock, which was generated from early passages and utilized for each experiment within 4 weeks of culture. Once a month, cell lines were tested for mycoplasma using the kit Lonza #LT07-118 ensuring that all the experiments using cell lines in this article were mycoplasma free. Luciferase-expressing-Capan-2 (Capan-2LUC) and -A549 cells (A549LUC) were obtained by transduction MOI5 of lentivirus particles encoding luciferase-GFP obtained using construct pTRIP-MND-Luciferase-hPGK-EGFP (Addgene, kindly provided by Didier Busso, CEA Fontenay-aux-Roses, France).

Cell transfection. Cells were transfected by nucleofection using the Human T Cell Nucleofector solution (Lonza, VPA-1002) and program U-014 of the Nucleofector AMAXA. For CRISPR, 2×106 cells were nucleofected with 75 pmol of Cas9 protein (Thermo Fisher, #A36499) and one RNA guide targeting the FOXO1 gene (Thermo Fisher, #sgRNACRISPR889854_SGM Foxo1) at a 1:1 molar ratio. To induce FOXO1 expression, 5×106 cells were transfected with 5 μg DNA of the pEGFP-FOXO1-T24A-S256A-S319A-H215R plasmid (Nagashima et al., 2010) or pEGFP-N1 (clontech) as control. Jurkat T cells (5×106 in RPMI 1640 medium) were electroporated at 260 V, 1000 μF in 4-mm polycarbonate cuvettes (Eurogentec) with 5 μg of the GFP, the triple mutant T24A/S256A/S319A FOX01-GFP (FOXO1-TM-GFP) or the FOXO1-TM-GFP DNA binding mutant plasmids (FOXO1-TM-H215R-GFP) (Fabre et al. 2008). Four hours later, AS1842856 was added or not in the culture. At 48 hours, CD62L expression was analyzed by cytometry.

Flow cytometry. Cells were washed and stained with antibodies diluted in PBS (Thermo Fisher Scientific; cat #10010001) supplemented with 0.1% BSA (Sigma-Aldrich, cat #A7030-500G) for 20 minutes at 4° C. After wash with PBS, cells were immediately analyzed by flow cytometry. For intracellular staining, cells were fixed with 4% paraformaldehyde (PFA) for 10 minutes at room temperature, permeabilized using a buffer containing 0.1% Saponin (Sigma-Aldrich, 84510) in PBS and stained after two PBS washes using antibodies diluted in the same saponin buffer for 30 minutes at 4° C. After two washes, cells were run on BD LSRII (BD Biosciences) and analyzed using FlowJo software.

Viability assay. Cells were collected and propidium iodide was added (10 μg/ml, Invitrogen). Samples were run on FACSCalibur (Becton Dickinson) and analyzed using FlowJo software.

Cytotoxicity assays. CD8⁺ T cells were purified from T^{AS} or untreated T cells by negative selection with EasySep™ Human CD8⁺ T Cell Isolation Kit (Stem Cell, #17953). The cytotoxic activity was measured by a conventional 4 hours 51Cr-release assay using triplicate cultures in round-bottom 96-wells plates (Falcon). Effector (E): Target (T) ratios were 30:1, 10:1, 3:1 and 1:1 on 3000 target cells/well. Percent specific cytotoxicity was calculated conventionally (Echchakir et al. 2000). The FcR-positive P815 murine cells were incubated with OKT3 antibody during 1 hour at 37° C. and used as target in redirected cytotoxicity assay.

Proliferation assay. In vitro proliferation assay of primary T cells was assessed by dilution of Cell Trace Fluorescent Green kit (CFSE, Invitrogen, cat #C34554). After two washes in RPMI 1640 GlutaMAX medium, cells were resuspended at 4×10⁶ cells per ml in a 5 μM Cell Trace Fluorescent Green solution. Cells were incubated in dark at 37° C. for 20 minutes. After the loading, cells were washed with a volume of hot RPMI medium supplemented with 10% Human AB serum, penicillin and streptomycin (50U/ml and 50 μg/m respectively) corresponding to 5 times the loading volume. Cell suspension was collected and dispensed into 96-wells round-bottom plates (2×10⁵ cells per well) and stimulated using anti-CD3/CD28 coated-beads Dynabeads (Invitrogen, #11131D) for 3 days. Samples were run on BD LSR Fortessa and analyzed using FlowJo software. For in vivo proliferation assay, T^{AS} cells were labeled by dilution of CFSE as for in vitro proliferation assay described above. Then 10⁷ CFSE⁺ cells per immunodificient NSG mouse (NOD.Cg-Prkde^{scid} I12rg^{tm1WjI}/SzJ mouse, cat #JAX: 005557, Charles River) resuspended in 100 μL PBS supplemented by 10% FBS were intravenously injected in the retro-orbital vein. 5, 9 or 12 days after injection, spleens were collected to be minced and meshed on the top of a 40 μm cell strainer (Thermo Fisher, #22363547). Cell suspensions were run on FACSCalibur. Some CFSE⁺ cells were maintained in culture to serve as control from the day of injection to the end of the experiment. At 5, 9 or 12 days following the beginning of the culture, cells suspension were run on FACSCalibur (Becton Dickinson) at the same time as cells from spleens. Data were analyzed using FlowJo software.

ImageStream flow cytometry. Cells were washed once in cold PBS and fixed for 20 minutes on ice in cytofix/cytoperm solution (Invitrogen #00-5523-00). Cells were then stained with Phalloidin-TRITC for 30 minutes at room temperature (Life Technologies, #R415). Flow cytometry was performed on an ImageStreamX MKII high-speed imaging flow cytometer (Amnis Corporation) and shape deformation was evaluated by aspect ratio which is the minor axis divided by the major axis with an IDEAS Analysis Software (Amnis Corporation).

Motility assay. Human pancreatic Capan-2 tumors were established by transplantation of 10⁷ cells injected subcutaneously into the flanks of immunodeficient NSG mice. Two weeks later, mice were sacrificed, and tumors were isolated and embedded in 8% low-gelling-temperature agarose (Sigma-Aldrich; cat #A0701-25G) prepared in PBS. Tumor slices of 400 μm thickness were cut with a vibratome (Leica VT1200S vibratome, RRID: SCR_018453) in a bath of ice-cold PBS. Slices were stained using anti-human EpCAM and anti-mouse podoplanin antibodies (Table. Sup1) for 20 minutes at 37° C. by using a 0.4-μm organotypic culture inserts (Merck Millipore; cat #PICM03050). Then, slices were washed in PBS. 5×10⁶ T cells per treatment condition (vehicle or AS1842856) were washed twice with PBS, then labeled with 100 nM Calcein Green (Invitrogen, cat #C34852) or Calcein Red-Orange (Invitrogen, cat #C34851) in PBS for 20 minutes at 37° C. After one wash in the motility medium consisting of RPMI 1640 GlutaMAX supplemented with 0.5% of BSA, 1 mM Sodium Pyruvate and 10 mM HEPES (Gibco, cat #15630-056), cells from the two treatment conditions were mixed at 1:1 ratio and resuspended together in 100 μl of motility medium. 30 μL of the mix were put onto each slice and incubated for 20 minutes at 37° C. by using a 0.4-μm organotypic culture inserts. After a wash in PBS, slices were transferred into a 35 mm Petri dish (containing the motility medium as described above) and dynamic imaging of T cells on tumor slices was performed (10× objective, binning=2, 20 timepoints each 30 seconds, 15 μm z-stack) as previously described (Kantari-Mimoun et al. 2021). Migration of T cells on slices was measured using the plugin TrackMate of ImageJ software. Software parameters were set as follows: LoG detector (estimated object diameter 10 μm, threshold 50000), no filter on spots, simple LAP tracker (linking max distance 20 μm, gap-closing max distance 20 μm, gap-closing max frame gap 3). Only tracks containing 5 or more spots were considered for analysis.

Calcium Measurements. T cells were incubated for 20 minutes at 37° C. with 1.5 μM Fura-2/AM (Molecular Probes, F1225). Experiments were performed at 37° C. in mammalian saline buffer (140 mM NaCl, 5 mM KCl, 1 mM CaCl2, 1 mM MgCl2, 20 mM HEPES, 11 mM glucose). Calcium measurements by spectrofluorimetry were performed as previously described (Conche et al. 2009) with a Cary Eclipse spectrofluorimeter (Varian) (excitation: 340 and 380 nm; emission: 510 nm).

Western blot analysis. Protein expression levels were analyzed by Western blot as described (Froehlich et al. 2016). Blotting with primary was followed by goat-anti-mouse- or goat-anti-rabbit-HRP (Jackson ImmunoReseach) incubation and ECL revelation (GE Healthcare, #RPN2106).

Lentivirus production. Vesicular stomatitis virus glycoprotein (VSV-G)-pseudotyped retroviral vectors were generated by transfecting HEK 293T cells as previously described (Berger et al. 2011). The plasmids used were pVSV-G (Plasmid #8454, Addgene), lentiviral packaging plasmid pCMVR8.74 coding HIV GAG/POL/REV (Plasmid #22036, Addgene) and lentiviral transfer vector plasmids shown in Fig. Sup1. CAR sequence is composed, downstream of a signal peptide, of scFv directed against EGFR derived from nimotuzumab sequence (IMGT/2Dstructure-DB INN 8545H, 8545L), CD8 hinge and transmembrane domain (194-248 Aa, GenBank: AAH25715.1), 4-1BB costimulatory domain (214-255 Aa, GenBank: AAX42660.1), CD3z signaling domain (52-163 Aa, GenBank: NP_000725.1) and is associated to GFP by IRES sequence or P2A sequence. All viral stocks were titrated by infecting 8×10⁴ MT4R5 cells with a range of the produced lentivirus and by analyzing the required volume to obtain 50% of GFP⁺ cells after 3 days of infection.

Generation of CAR-T cells. 2×10⁶ T cells were cultivated in a 24-wells plate in TEXMACS medium (Miltenyi, #130-097-19) supplemented with 10 ng/ml of human IL-7 (Miltenyi, #130-095-362) and 10 ng/ml of human IL-15 (Miltenyi, #130-095-764) and activated by TransAct (Miltenyi, #130-111-16) at a 1/100 dilution. Activated T cells were transduced 48 hours after stimulation using MOI2 of the lentivirus stock. GFP expression was analyzed by flow cytometry 3 days after transduction. Transduction efficiency with EGFR CAR was between 50% and 85% of positive cells.

Generation of CAR-T^{AS} cells. T lymphocytes from healthy donor blood were cultured in complete RPMI containing 10% Human AB serum, penicillin and streptomycin (50U/ml and 50 μg/ml respectively) at a concentration of 3×10⁶ cells/ml in the presence of 500 nM of AS1842856. At day 7, T^{AS} cells were transduced with MOI2 of the lentivirus stock. GFP expression was analyzed by flow cytometry.

In vivo assay. 6-8 weeks old NSG mice were used for in vivo assays. For the Capan-2 model, 5×10⁶ luciferase-expressing Capan-2 cells (Capan-2^{luc}) were subcutaneously injected in the shaved right flank of each mouse. 7 days after injection, CAR-T cells were injected intravenously in the retro-orbital vein. For the A549 model, 3×10⁶ A549^{LUC} were intravenously injected in the tail vein. 3 days after injection, 2×10⁵ CAR-T cells or CAR-T^{AS} cells were injected intravenously in the retro-orbital vein. The follow up of tumor growth was assessed once a week by bioluminescence detection by using Photon Imager RT (Biospacelab) or with a caliper when specified. For Capan-2 model only, at day 40 post CAR-T cells injection, tumors were observed by ultrasound scan using VEVO2100 device (Visualsonics). At the end of the experiment, mice were sacrificed, and tumors were collected and weighed.

Statistical Analysis. The statistical tests used for sample comparison are specified in the figure legends (*p<0.05; **p<0.01; ***p<0.001; ns: not significant). They were performed with GraphPad Prism software.

### Results

### FOXO1 Inhibition Increases Activity of Cytotoxic T Cells

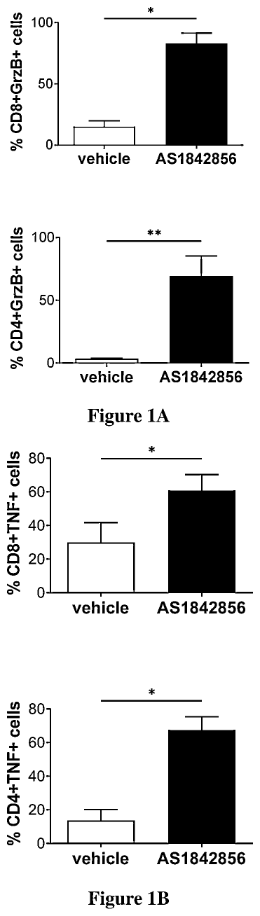

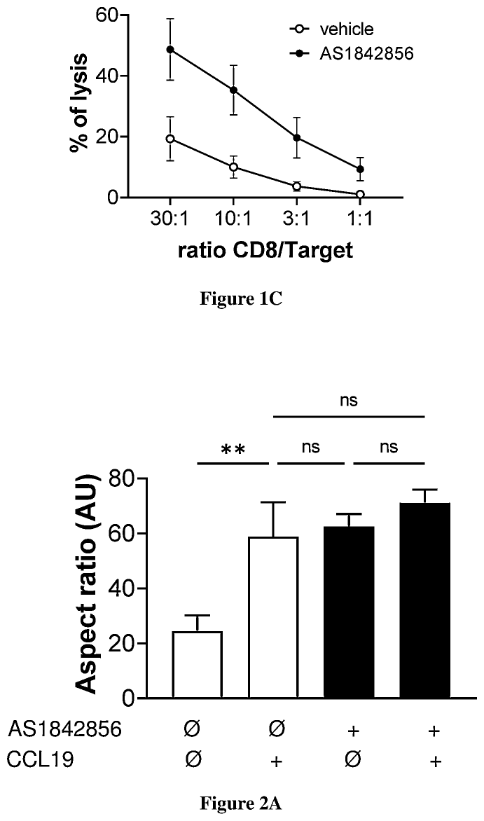

The basis of CAR-T cell immunotherapy is the ability of these cells to kill tumor cells. Their cytotoxic activity is therefore essential. We therefore looked at the consequences of FOXO1 inhibition with AS1842856 on the cytotoxic activity of T cells. As previously published, we observed that granzyme B, a key molecule to induce lysis of target cells, is upregulated in the presence of the FOXO1 inhibitor in the CD8 subset (Jeng et al., 2018; Roux et al., 2019). Surprisingly this proved to be also true for CD4 T cells (FIG. 1A). Beyond granzyme B, an increased cytotoxic activity could also result from the production of pro-inflammatory cytokines such as TNF-α. We therefore compared the percentage of cells expressing TNF-α by intracellular labeling in T cells treated or not with AS1842856. We found that inhibition of FOXO1 significantly increased TNF-α expression (FIG. 1B). These results were confirmed using the MSD assay to measure the amount of TNF-α secreted in the supernatant of T cells treated with AS1842856 (data not shown). Of note, we found that other inflammatory cytokines, such as IL-10 and GM-CSF, but not IFN-α, were also secreted in increased amounts by T^{AS} cells (data not shown). These characteristics were correlated to a stronger cytotoxic activity toward P815 target cells from CD8 T^{AS} cells compared to untreated T cells as measured in redirected lysis experiments (FIG. 1C). Thus, inhibition of FOX01 potentiates killing of a tumor target, a fundamental function of CAR-T cells.

To confirm the role of FOXO1 in the AS1842856-mediated increase of cytotoxic functions, we analyzed the consequences of Foxo1 gene invalidation by CRISPR on granzyme B expression. We first observed that Foxo1 invalidation, as AS1842856 treatment, leads to a decrease of CD62L expression, since it is a transcriptional target of FOXO1 (Fabre et al. 2008). Under the same conditions, while AS1842856 induces a more than two-fold increase of granzyme B expression, Foxo1 invalidation does not induce any variation in granzyme B (data not shown). So, the increase of granzyme expression level by AS1842856 could not be recapitulated by the absence of FOXO1. As AS1842856 leads to an increase of FOXO1 lacking the ability to interfere with its specific DNA binding sites, we analyzed the consequences of overexpression of the FOXO1 H215R mutant. This mutant form of FOXO1 mimics AS1842856 treatment as it encodes a constitutively nuclear form of FOXO1 unable to bind to its DNA binding sites. Overexpression of this FOXO1 mutant induced not only a clear decrease of CD62L but also a strong increase in granzyme B expression (data not shown). We also observed after transfection of Jurkat T cell line that FOXO1 H215R mutant induced a similar downregulation of CD62L expression, that could not be further decreased by AS1842856 (data not shown). Thus, AS1842856 allows to dissociate the different mechanisms of FOX01 transcription regulation by inhibiting only its bona fide transcription factor activity, resulting from its interaction with its DNA binding sites.

### FOXO1 Inhibition Promotes T Cells Motility.

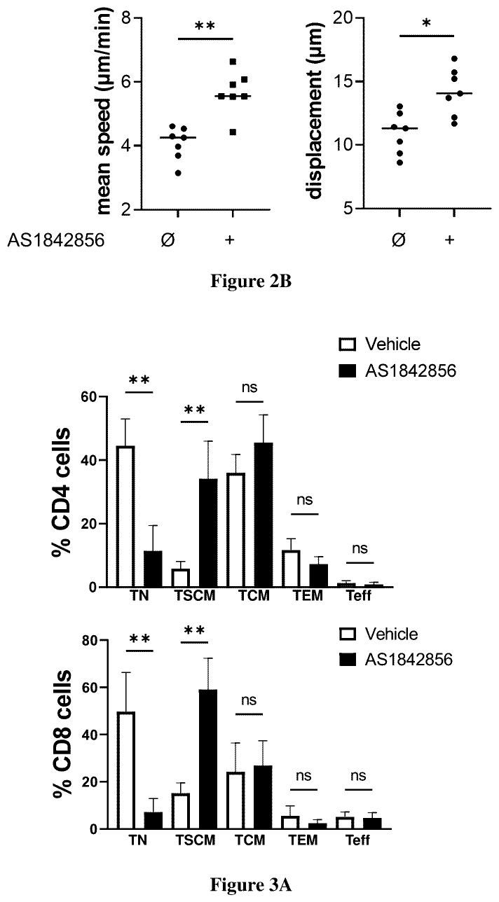

Another limitation of CAR-T cells therapy, especially in the context of solid tumors, is the ability of these cells to penetrate the tumor bed and move within the tumor (Majzner & Mackall, 2019). As we have shown that some FOXO1 target genes regulate T cell mobility (Fabre et al., 2008; Megrelis et al., 2018; Rougerie et al., 2013), we next analyzed whether inhibition of FOXO1 could affect the movement of T cells. We first observed that T^{AS} cells spontaneously adopted a strong shape alteration, typical of polarized cells, seen through a decrease in their aspect ratio, which becomes clearly inferior when compared to that of untreated T cells (FIG. 2A). As this T cell deformation is usually relied on chemokine response, we compared cell polarization upon CCL19 stimulation on AS1842856-treated T cells or not treated ones. We observed that upon chemokine stimulation, T cells and T^{AS} cells presented a comparable aspect ratio. Overall, these results show that inhibition of FOXO1 induces spontaneous cell polarization comparable to that obtained by stimulation with chemokines.

As differentiation of T cells is often associated with major reprogramming of chemokine receptor expression, we analyzed by flow cytometry the consequences of AS1842856 treatment on some of them. We show that the modulation of their membrane expression by AS1842856 is specific to each chemokine receptor. Thus, while there is a significant decrease in CXCR4, inhibition of FOXO1 induces an increase in CCR4, CX3CR1 and CCR6 (data not shown), three receptors that have been described to promote T cell migration within the tumor microenvironment.

Because polarity establishment is associated with the capacity to migrate, we next aimed at testing the consequences of FOXO1 inhibition on T cell migration. We used a device developed by Asperti-Boursin et al. to monitor the motile behavior in live tumor slices (Asperti-Boursin et al., 2007). For these experiments, Capan-2 cells were implanted subcutaneously into NSG mice. Two weeks later, tumors were sliced with a vibratome, and T cells were plated onto fresh slices. As shown in FIG. 2C, the inhibition of FOXO1 endowed T cells with a greater capacity to move in the tumor microenvironment, as illustrated by the increase of their speed and the length of their course. The increased motility of TAS cells was also observed in an orthotopic tumor model resulting from intravenous injection of the A549 cell line from lung carcinoma (data not shown). So, inhibition of FOXO1 induces a T cell polarization comparable to that triggered by chemokines as well as an increased T cell motility within tumors.

### FOXO1 Inhibition Induces the Acquisition of a Stem Cell Memory Phenotype.

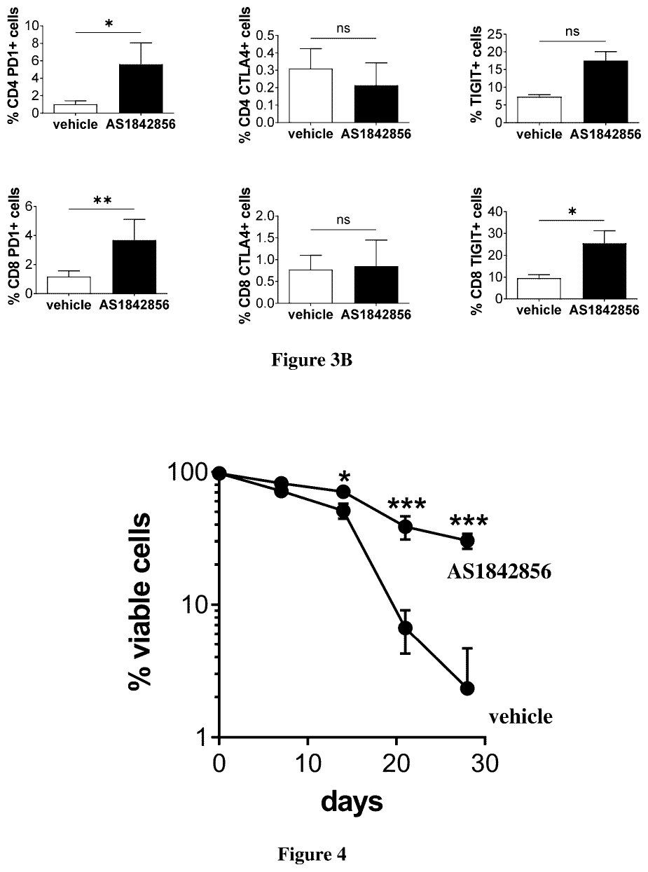

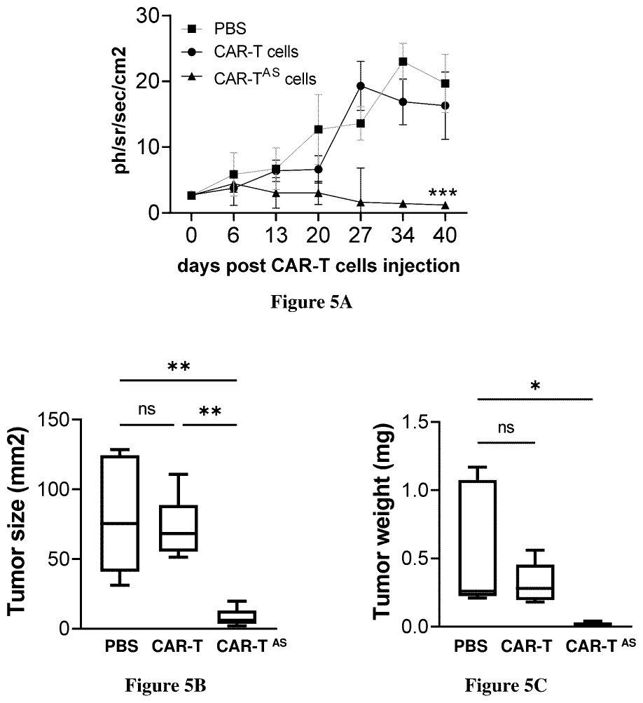

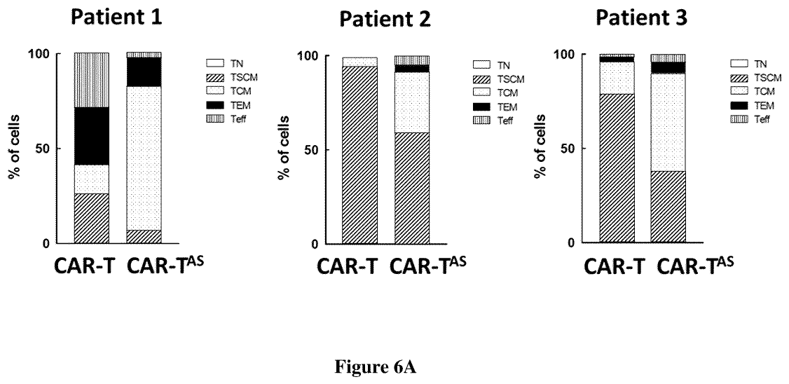

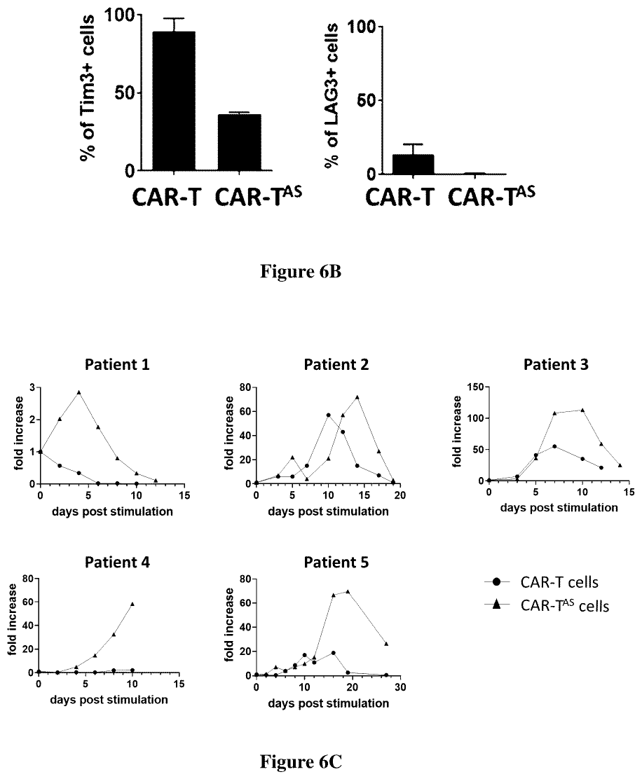

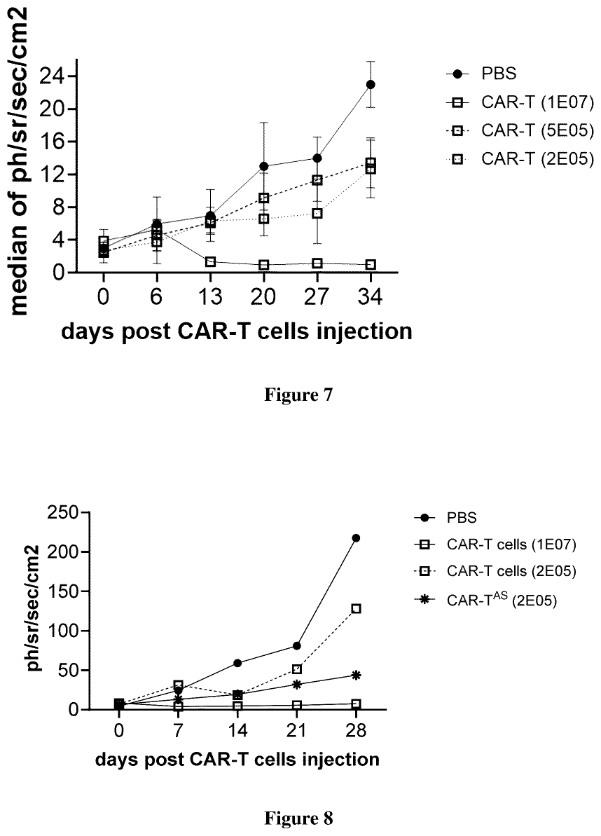

We have previously shown that FOXO1 inhibition induces an increase in metabolic activity, a phenotype known to be associated with the appearance of memory features (Jeng et al., 2017). We therefore analyzed by flow cytometry the different T cells subsets obtained after an in vitro treatment of human primary T cells with AS1842856 for 48 hours. As FoxO1 is involved in several CD4 subsets differentiation, we investigated the consequences of AS1842856 treatment on the expression of markers associated to Tfh (CXCR5+, BcL-6+), Th1 (T-bet+), Th2 (GATA3+) and Treg (FOXP3+) subsets and found no significant changes, except for Tfh that presents a slight increase after AS1842856 treatment (data not shown). Using a gating strategy shown in FIG. 3A, we quantified the relative proportion of naïve (T_{N}), stem cell memory (T_{SCM}), central memory (T_{CM}) and effector memory (T_{EM}) T cells. We observed that AS1842856 induced a profound modification of the differentiation state of T cells, with a sharp decrease of the naive subset (CD45RA+/CD27+/CD95−) and a strong increase of the T_{SCM} subset (CD45RA+/CD27+/CD95+), for both CD4 and CD8 T cells. Incidentally, this phenotypic analysis allowed us to observe that AS1842856 induces a decreased expression of CD8 (data not shown), as already reported for primed CD8 T cells in both humans and mice (Erard et al., 1993; Kambayashi et al., 2001; Maile et al., 2001), confirming the priming of T cells induced by FOXO1 inhibition. In T lymphocytes, differentiation can be associated with an increased expression of exhaustion markers, also known as immune checkpoints, that negatively regulate T cells effector functions. As shown in FIG. 3B, we observed a small but significant increase in PD1 expression in both CD8+ and CD4+ cells (left panel), with no more than 10% of cells expressing this marker. We detected no significant modification of CTLA-4 (middle panel), and a consistent increase of TIGIT, but reaching significance only in CD8+ T cells. Overall, this phenotypic analysis showed that the most noticeable events induced by FOXO1 inhibition in T cells are a sharp decrease in the naive subset correlated with a sharp increase of memory T cells, especially T_{SCM}, in both CD4+ and CD8+ T cells.

### T^{AS} Cells Display Functional Characteristics of Memory T Cells.

We then sought to assess whether the acquisition of this memory phenotype was accompanied with the acquisition of functional characteristics classically associated with memory T cells. We first compared the in vitro survival of primary T cells with or without FOXO1 inhibition. We observed a prolonged survival T^{AS} cells (FIG. 4), so that after 28 days of culture, 30%+/−4% of T^{AS} cells were still alive compared to only 2%+/−4% of untreated cells. This result corresponds to an increase in survival since under these culture conditions T cells do not proliferate (data not shown). Thus, AS1842856 not only exhibits no toxicity but also promotes T cell viability. Memory T cells have also been described to have a specific signaling signature upon TCR stimulation. Especially, calcium influx has been described to be lower in memory T cells compared to naive T cells (Adachi & Davis, 2011; Hall et al., 1999; Tanchot et al., 1998). We observed that the calcium influx induced upon TCR stimulation was less rapid and of a lower magnitude in AS1842856-treated T cells as compared to untreated cells (data not shown). In addition to the calcium response, phosphorylation of ERK and AKT signaling proteins upon TCR stimulation has also been described to be different between naive and memory T cells populations (Adachi & Davis, 2011; N. Jones et al., 2019; Kalland et al., 2011). The comparison of the kinetics of phosphorylation of ERK and AKT upon TCR stimulation of AS1842856-treated and untreated cells by western blot revealed a clear hypophosphorylation of AKT and hyperphosphorylation of ERK after TCR stimulation, including at steady state (data not shown). Thus, analyses of both calcium response and phosphorylation of ERK and AKT proteins, revealed that T cells treated with AS1842856 behaved as memory cells. Finally, we evaluated the consequences of FOXO1 inhibition on the proliferative capacities of T cells as it has been described that non fully differentiated memory cells like TSCM cells have greater proliferative (Gattinoni et al., 2011). For this aim, T cells treated or not with AS1842856 were stained with CFSE and then stimulated with anti-CD3/CD28 beads. Cell proliferation was followed for 60 hours. We found that T cells treated with AS1842856 proliferated as early as 40h after stimulation while 8h of additional stimulation was required for untreated cells (data not shown). This proliferative advantage is maintained over time since after 60h of stimulation, we still observed a greater number of CFSE dilution peaks with T cells treated with AS1842856 (data not shown). A dose-response curve showed that 48 hours post stimulation the ratio of percentage of cells in proliferation was identical for each bead/cell ratio for control and T^{AS} cells (data not shown). So, the proliferative advantage of FOXO1 inhibition did not alter the activation threshold of T^{AS} cells. Altogether, these results show that AS1842856 induces a clear phenotypic and functional transition from naive to memory T cells.

### CAR Transduction in Quiescent Cells Thanks to FOXO1 Inhibition.

In CAR-T cell production protocols, T cells are first stimulated in vitro to enable CAR expression into T cells. However, this ex vivo stimulation likely comes at the cost of cells exhaustion (Ghassemi et al., 2018). We therefore explored the possibility of introducing a CAR encoding vector into T^{AS} cells as we have previously shown that FOXO1 inhibition makes T cells permissive to infection (Roux et al., 2019). Since the CAR sequence is linked to the GFP sequence by an Internal Ribosome Entry Site (IRES), we monitored the expression of the chimeric receptor by analyzing GFP expression. In a first series of experiments, we observed that unlike the viruses used as controls, viruses encoding the CAR allowed to very poor transduction of T^{AS} cells. The absence of productive transduction of T^{AS} cells by viruses encoding CAR did not result from virus infectivity since pre-activated T cells were comparably transduced with the two types of viruses (data not shown). To characterize the absence of productive transduction, we explored the possibility that non expression could result from a latent infection as it was previously described (Brooks et al., 2003; Novis et al., 2013). So, we transduced T^{AS} cells with viruses encoding CAR, and then after three days of culture, transduced T^{AS} cells were stimulated with anti-3/28 beads. While T^{AS} cells only weakly express CAR just after transduction, activation induces a strong increase of CAR expression, suggesting that transduction of T^{AS} cells with CAR encoding particles results in silent infections. We first hypothesized that this lack of productive transduction was due to the promoter used. Indeed, in the retroviral vector use, the CAR was under the control of the EF1α promoter. This promoter has been previously described as weakly active in quiescent cells (Ho et al., 2021; S. Jones et al., 2009). We therefore created new vectors in which the CAR was under the control of MND or CMV promoters described to be active in quiescent T cells (Ho et al., 2021; S. Jones et al., 2009). We found that regardless of the promoter used, transduction of T^{AS} cells induces only a low CAR expression, whereas they induced expression comparable to the virus control in pre-activated T cells. We then explored the possibility that this lack of productive transduction of T^{AS} cells results from a low expression of GFP downstream IRES sequence. We therefore constructed retroviral vectors in which the CAR was linked to GFP via a P2A linker. Again, we found that CAR viruses were not able to induce CAR expression in T^{AS} cells. All these data leaded us to conclude that although we were unable to establish the cause of this low productive transduction efficacy of T^{AS} cells, we observed that it was strongly increased following the stimulation of T cells.

An immunodepletion step precedes CAR-T cell injection in clinical protocols, (Majzner & Mackall, 2019) and it has been shown that a phenomenon called homeostatic proliferation occurs when T cells are injected into immunocompromised hosts (Zwang & Turka, 2014). Thus, it is conceivable that CAR expression in T^{AS} cells could occur following T-cell activation and expansion in the host. To test this hypothesis, we first analyzed the proliferation of T^{AS} cells after injection in immunodeficient mice which mimic the aplastic state in patients. CFSE-labelled T^{AS} cells were injected into mice or maintained in vitro and we compared their proliferation (data not shown). We observed that T^{AS} cells proliferated in vivo as early as day 9 after injection, whereas the same cells did not proliferate in vitro. This result shows that T^{AS} cells undergo a homeostatic proliferation after in vivo injection. To further investigate whether this proliferation correlates with CAR expression, we injected CAR-T^{AS} cells and harvested the spleens 9 days after injection. The results obtained illustrated that the homeostatic proliferation is correlated with the CAR expression induction in CAR-T^{AS} cells in vivo (data not shown). These data allow us to consider that we have established a protocol where CAR expression was inducible by proliferation.

CAR-T^{AS} Cells are More Efficient than Classical CAR-T Cells to Eradicate Tumors In Vivo.

To investigate the potential benefit of this new protocol of CAR-T cells (CAR-T^{AS}) production, we used a classical model based on the regression of tumors formed by subcutaneously injected human tumor cells into immunodeficient mice followed by CAR-T cell injection. We used the Capan-2 cell line, which is known to express large amounts of the Epidermal Growth Factor Receptor (EGFR), and is a target of EGFR-targeted CAR (Guedan et al., 2018). Specifically (FIG. 5A), we subcutaneously injected 5×10⁶ luciferase expressing Capan-2 cells (Capan-2^{Luc}) into NSG mice. Seven days later, we injected EGFR-targeted CAR T cells in the orbital sinus and followed tumor growth by bioluminescence. In this experimental model, 10⁷ classical CAR-T cells are required to induce tumor regression. With 5×10⁵ CAR-T cells, tumors were only moderately controlled (FIG. 7). To ensure that FOXO1 inhibition did not exacerbate allogenic response of T^{AS} cells against Capan-2 tumor, we first compared tumor growth after T cells treated or not with AS1842856. Injection of 10⁶ T^{AS} cells did not lead to tumor regression. We next compared tumor growth after injection of 2×10⁵ classical CAR-T cells or 2×10⁵ CAR-T^{AS} cells. As shown in FIG. 5, we observed that in this setting, classical CAR-T cells were not able to control tumor growth whereas CAR-T^{AS} cells induced a sustainable tumor regression. This result was confirmed by tumor surface measure by ultrasound scan at day 40 (FIG. 5B) and by tumor weight at the end of the experiment (FIG. 5C). It should be noted that an allogenic response of T^{AS} cells against Capan-2 tumor can be excluded in the observed effect since injection of 106 T^{AS} cells did not lead to any tumor regression nor mice weight loss (data not shown). To extend the applicability of CAR-T^{AS} cells, we compared the antitumor effect of CAR-T cells and CAR-T^{AS} cells after intravenous injection of A549 cells, derived from a human lung carcinoma tumor. This injection route (i.v) allows an orthotopic localization as the engraftment of the tumor occurs in lungs (FIG. 8). Again, in this model, CAR-T^{AS} cells demonstrated a much stronger effect than classical CAR-T cells. Therefore, we concluded that CAR-T^{AS} cells were more efficient than classical CAR-T cells to treat solid tumors.

CAR-T^{AS} Generated from Patients' Cells Expand More than Classical CAR-T Cells

Although these results were encouraging, they were performed using T cells from healthy donors. But, it is now known that the patient's immune system is impaired by the repeated therapies that precede the CAR-T cell therapy, but also by the pathology itself. So, it has been largely published that T cells from cancer patients are not equivalent to T cells from healthy donors and this in several cancer diseases (Hoffmann et al., 2017; Metelo et al., 2022). Thus, to validate the advantage of CAR-TAS cells in a clinical context, we realized a series of experiences on T cells collected from patients included in a CAR-T cells therapeutic protocol. Two patients had a primary mediastinal lymphoma refractory to second line therapy, one patient had a high-grade large B cell lymphoma refractory to third line therapy, two patients had mantle cell lymphoma in relapse after three lines of treatment and refractory to three lines of treatment respectively, including one burton tyrosine kinase inhibitor for each and an autologous stem cell transplantation procedure for one of them. We compared the phenotypic and functional characteristics of classical CAR-T cells and CAR-TAS cells. We first validated the feasibility to obtain CAR-TAS and CAR-T cells from patients' cells with comparable efficiencies (data not shown). Next, we observed in FIG. 6A, that the classical protocol to obtain CAR-T cells allowed to give rise to more cells presenting a TSCM-specific phenotype than the AS1842856 protocol did for all the 3 patients' cells tested even if patient 1 had globally more differentiated T cells in CAR-T cells than in CAR-TAS cells. However, since TCR stimulation induces expression of memory markers, we could not state that the TSCM phenotype observed for classical CAR-T cells corresponds to memory cells per se. In addition, immune checkpoints analysis revealed that CAR-TAS cells present a less exhausted phenotype than CAR-T cells with a reduced expression of Tim3 and LAG-3 (FIG. 6B). One parameter of a favorable prognosis for the success of the therapy is the capacity of the cells to early expand in the patient's body after infusion (Majzner et al., 2019; Fraietta et al., 2018). Thus, to mimic this expansion, we stimulated in vitro by TransAct the different CAR-T cells at the end of their respective manufacturing protocol and we counted the cells every two days. The CAR-TAS cells from all patients exhibited a better proliferative capacity than the classical CAR-T cells although CAR-TAS cells from the patient 2 have a delay compared to CAR-T cells (FIG. 6C). These results show that CAR-TAS cells can be readily obtained from CAR-eligible patients, and have retained better expansion properties than classical CAR-T cells.

### CONCLUSION

In this study, the inventors showed that AS1842856 as inhibitor of FOXO1 caused on human T cells purified from healthy donors and after a few days of culture in the absence of any growth factor (cytokines, etc.), a substantial increase in their metabolic activity that correlates with acquisition of phenotypic and functional characteristics of activated/memory T cells. More specifically, they found that AS1842856 induces an increase in the production of granzyme B in CD8+ T cells but also in CD4+ T cells resulting in a potentiation of the cytotoxic activity of human CD8+ T primary lymphocytes. Thanks to permissivity induced by FOXO1 inhibition, they showed that a simple pharmacological treatment during ex vivo culture can generate CAR-T cells lacking the exhausted characteristics of classical CAR-T cells. They observed that inhibition of FOXO1 by AS1842856 pharmacological agent not only allows quiescent T cells infection but also the acquisition of phenotypic and functional characteristics leading to a strong increase of CAR-T cell antitumoral activity. Notably, they showed for the first time that the inhibition of FOXO1 potentiates the ability to induce lysis of a target cell thanks to the increased expression of TNF-α and other inflammatory cytokines, induces spontaneous cell polarization comparable to that obtained by stimulation with chemokines and thus improves the motility of the cells, induces a sharp increase of memory T cells, improves the proliferation of the T cells and allows to obtain more efficient CAR-T cells to treat solid tumors than classical CAR-T cells obtained with the known protocol. They also show that T cell obtained with their protocol undergo a homeostatic proliferation after in vivo injection which induces the CAR expression. The results obtained with T cells from patients allowed the generation of CAR-T cells with an elevated potential of expansion likely to improve the current therapy in clinic which remains limited to certain tumors.

### REFERENCES

Throughout this application, various references describe the state of the art to which this invention pertains. The disclosures of these references are hereby incorporated by reference into the present disclosure.
Adachi, K., & Davis, M. M. (2011). T-cell receptor ligation induces distinct signaling pathways in naive vs. antigen-experienced T cells. Proceedings of the National Academy of Sciences of the United States of America, 108 (4), 1549-1554. https://doi.org/10.1073/pnas.1017340108
Amara A., Vidy A., Boulla G., Mollier K, Garcia-Perez J, Alcamí J, Blanpain C, Parmentier M, Virelizier J L, Charneau P, Arenzana-Seisdedos F. (2003). G protein-dependent CCR5 signaling is not required for efficient infection of primary T lymphocytes and macrophages by R5 human immunodeficiency virus type 1 isolates. Journal of Virology, 77 (4): 2550-8. doi: 10.1128/jvi.77.4.2550-2558.2003.Asperti-Boursin, F., Real, E., Bismuth, G., Trautmann, A., & Donnadieu, E. (2007). CCR7 ligands control basal T cell motility within lymph node slices in a phosphoinositide 3-kinase-independent manner. The Journal of Experimental Medicine, 204 (5), 1167-1179. https://doi.org/10.1084/jem.20062079
Bowers, J. S., Majchrzak, K., Nelson, M. H., Aksoy, B. A., Wyatt, M. M., Smith, A. S., Bailey, S. R., Neal, L. R., Hammerbacher, J. E., & Paulos, C. M. (2017). PI3K8 Inhibition Enhances the Antitumor Fitness of Adoptively Transferred CD8 (+) T Cells. Frontiers in Immunology, 8, 1221. https://doi.org/10.3389/fimmu.2017.01221
Brooks, D. G., Arlen, P. A., Gao, L., Kitchen, C. M. R., & Zack, J. A. (2003). Identification of T cell-signaling pathways that stimulate latent HIV in primary cells. Proceedings of the National Academy of Sciences of the United States of America, 100 (22), 12955-12960. https://doi.org/10.1073/pnas.2233345100
Crompton, J. G., Sukumar, M., Roychoudhuri, R., Clever, D., Gros, A., Eil, R. L., Tran, E., Hanada, K.-I., Yu, Z., Palmer, D. C., Kerkar, S. P., Michalek, R. D., Upham, T., Leonardi, A., Acquavella, N., Wang, E., Marincola, F. M., Gattinoni, L., Muranski, P., . . . . Restifo, N. P. (2015). Akt inhibition enhances expansion of potent tumor-specific lymphocytes with memory cell characteristics. Cancer Research, 75 (2), 296-305. https://doi.org/10.1158/0008-5472.CAN-14-2277
Echchakir, H., Vergnon, I., Dorothée, G., Grunenwald, D., Chouaib, S., & Mami-Chouaib, F. (2000). Evidence for in situ expansion of diverse antitumor-specific cytotoxic T lymphocyte clones in a human large cell carcinoma of the lung. International Immunology, 12 (4), 537-546. https://doi.org/10.1093/intimm/12.4.537
Erard, F., Wild, M. T., Garcia-Sanz, J. A., & Le Gros, G. (1993). Switch of CD8 T cells to noncytolytic CD8-CD4-cells that make TH2 cytokines and help B cells. Science (New York, N.Y.), 260 (5115), 1802-1805. https://doi.org/10.1126/science.8511588
Fabre, S., Carrette, F., Chen, J., Lang, V., Semichon, M., Denoyelle, C., Lazar, V., Cagnard, N., Dubart-Kupperschmitt, A., Mangeney, M., Fruman, D. A., & Bismuth, G. (2008). FOXO1 regulates L-Selectin and a network of human T cell homing molecules downstream of phosphatidylinositol 3-kinase. Journal of Immunology (Baltimore, Md.: 1950), 181 (5), 2980-2989.
Fraietta J A, Lacey S F, Orlando E J, Pruteanu-Malinici I, Gohil M, Lundh S, et al. Determinants of response and resistance to CD19 chimeric antigen receptor (CAR) T cell therapy of chronic lymphocytic leukemia. Nat Med. United States; 2018; 24:563-71. Froehlich, J., Versapuech, M., Megrelis, L., Largeteau, Q., Meunier, S., Tanchot, C., Bismuth, G., Delon, J., & Mangeney, M. (2016). FAM65B controls the proliferation of transformed and primary T cells. Oncotarget, 7 (39), 63215-63225. https://doi.org/10.18632/oncotarget.11438
Gattinoni, L., Lugli, E., Ji, Y., Pos, Z., Paulos, C. M., Quigley, M. F., Almeida, J. R., Gostick, E., Yu, Z., Carpenito, C., Wang, E., Douek, D. C., Price, D. A., June, C. H., Marincola, F. M., Roederer, M., & Restifo, N. P. (2011). A human memory T cell subset with stem cell-like properties. Nature Medicine, 17 (10), 1290-1297. https://doi.org/10.1038/nm.2446
Ghassemi, S., Durgin, J. S., Nunez-Cruz, S., Patel, J., Leferovich, J., Pinzone, M., Shen, F., Cummins, K. D., Plesa, G., Cantu, V. A., Reddy, S., Bushman, F. D., Gill, S. I., O'Doherty, U., O'Connor, R. S., & Milone, M. C. (2022). Rapid manufacturing of non-activated potent CAR T cells. Nature Biomedical Engineering, 6 (2), 118-128. https://doi.org/10.1038/s41551-021-00842-6
Ghassemi, S., Nunez-Cruz, S., O'Connor, R. S., Fraietta, J. A., Patel, P. R., Scholler, J., Barrett, D. M., Lundh, S. M., Davis, M. M., Bedoya, F., Zhang, C., Leferovich, J., Lacey, S. F., Levine, B. L., Grupp, S. A., June, C. H., Melenhorst, J. J., & Milone, M. C. (2018). Reducing Ex Vivo Culture Improves the Antileukemic Activity of Chimeric Antigen Receptor (CAR) T Cells. Cancer Immunology Research, 6 (9), 1100-1109. https://doi.org/10.1158/2326-6066.CIR-17-0405
Guedan, S., Posey, A. D. J., Shaw, C., Wing, A., Da, T., Patel, P. R., McGettigan, S. E., Casado-Medrano, V., Kawalekar, O. U., Uribe-Herranz, M., Song, D., Melenhorst, J. J., Lacey, S. F., Scholler, J., Keith, B., Young, R. M., & June, C. H. (2018). Enhancing CAR T cell persistence through ICOS and 4-1BB costimulation. JCI Insight, 3 (1). https://doi.org/10.1172/jci.insight.96976
Hall, S. R., Heffernan, B. M., Thompson, N. T., & Rowan, W. C. (1999). CD4+CD45RA+ and CD4+CD45RO+ T cells differ in their TCR-associated signaling responses. European Journal of Immunology, 29 (7), 2098-2106. https://doi.org/10.1002/(SICI) 1521-4141 (199907) 29:07<2098:: AID-IMMU2098>3.0.CO; 2-B
Ho, J.-Y., Wang, L., Liu, Y., Ba, M., Yang, J., Zhang, X., Chen, D., Lu, P., & Li, J. (2021). Promoter usage regulating the surface density of CAR molecules may modulate the kinetics of CAR-T cells in vivo. Molecular Therapy. Methods & Clinical Development, 21, 237-246. https://doi.org/10.1016/j.omtm.2021.03.007
Hoffmann J-M, Schubert M-L, Wang L, Hückelhoven A, Sellner L, Stock S, et al. Differences in Expansion Potential of Naive Chimeric Antigen Receptor T Cells from Healthy Donors and Untreated Chronic Lymphocytic Leukemia Patients. Front Immunol. Switzerland; 2017; 8:1956.
Izsvák Zsuzsanna et al. Translating Sleeping Beauty transposition into cellular therapies: Victories and challenges. Bioessays. 2010 September; 32 (9): 756-767.
Jeng, M. Y., Hull, P. A., Fei, M., Kwon, H.-S., Tsou, C.-L., Kasler, H., Ng, C.-P., Gordon, D. E., Johnson, J., Krogan, N., Verdin, E., & Ott, M. (2017). Metabolic reprogramming of human CD8+ memory T cells through loss of SIRT1. The Journal of Experimental Medicine. https://doi.org/10.1084/jem.20161066
Jeng, M. Y., Hull, P. A., Fei, M., Kwon, H.-S., Tsou, C.-L., Kasler, H., Ng, C.-P., Gordon, D. E., Johnson, J., Krogan, N., Verdin, E., & Ott, M. (2018). Metabolic reprogramming of human CD8 (+) memory T cells through loss of SIRT1. The Journal of Experimental Medicine, 215 (1), 51-62. https://doi.org/10.1084/jem.20161066
Jones, N., Vincent, E. E., Cronin, J. G., Panetti, S., Chambers, M., Holm, S. R., Owens, S. E., Francis, N. J., Finlay, D. K., & Thornton, C. A. (2019). Akt and STAT5 mediate naïve human CD4+ T-cell early metabolic response to TCR stimulation. Nature Communications, 10 (1), 2042. https://doi.org/10.1038/s41467-019-10023-4
Jones, S., Peng, P. D., Yang, S., Hsu, C., Cohen, C. J., Zhao, Y., Abad, J., Zheng, Z., Rosenberg, S. A., & Morgan, R. A. (2009). Lentiviral vector design for optimal T cell receptor gene expression in the transduction of peripheral blood lymphocytes and tumor-infiltrating lymphocytes. Human Gene Therapy, 20 (6), 630-640. https://doi.org/10.1089/hum.2008.048
Kalland, M. E., Oberprieler, N. G., Vang, T., Taskén, K., & Torgersen, K. M. (2011). T cell-signaling network analysis reveals distinct differences between CD28 and CD2 costimulation responses in various subsets and in the MAPK pathway between resting and activated regulatory T cells. Journal of Immunology (Baltimore, Md.: 1950), 187 (10), 5233-5245. https://doi.org/10.4049/jimmunol.1101804
Kambayashi, T., Assarsson, E., Chambers, B. J., & Ljunggren, H. G. (2001). IL-2 down-regulates the expression of TCR and TCR-associated surface molecules on CD8 (+) T cells. European Journal of Immunology, 31 (11), 3248-3254. https://doi.org/10.1002/1521-4141 (200111) 31:11<3248:: aid-immu3248>3.0.co; 2-3
Kantari-Mimoun, C., Barrin, S., Vimeux, L., Haghiri, S., Gervais, C., Joaquina, S., Mittelstaet, J., Mockel-Tenbrinck, N., Kinkhabwala, A., Damotte, D., Lupo, A., Sibony, M., Alifano, M., Dondi, E., Bercovici, N., Trautmann, A., Kaiser, A. D., & Donnadieu, E. (2021). CAR T-cell Entry into Tumor Islets Is a Two-Step Process Dependent on IFNγ and ICAM-1. Cancer Immunology Research, 9 (12), 1425-1438. https://doi.org/10.1158/2326-6066.CIR-20-0837
Klebanoff, C. A., Crompton, J. G., Leonardi, A. J., Yamamoto, T. N., Chandran, S. S., Eil, R. L., Sukumar, M., Vodnala, S. K., Hu, J., Ji, Y., Clever, D., Black, M. A., Gurusamy, D., Kruhlak, M. J., Jin, P., Stroncek, D. F., Gattinoni, L., Feldman, S. A., & Restifo, N. P. (2017). Inhibition of AKT signaling uncouples T cell differentiation from expansion for receptor-engineered adoptive immunotherapy. JCI Insight, 2 (23). https://doi.org/10.1172/jci.insight.95103
Li, H., & Chen, B. K. (2019). Variable infectivity and conserved engagement in cell-to-cell viral transfer by HIV-1 Env from Clade B transmitted founder clones. Virology, 526, 189-202. https://doi.org/10.1016/j.virol.2018.10.016
Lee et al., FOXO1 inhibition synergizes with FGF21 to normalize glucose control in diabetic mice. Molecular metabolism, 2021
Maile, R., Wang, B., Schooler, W., Meyer, A., Collins, E. J., & Frelinger, J. A. (2001). Antigen-specific modulation of an immune response by in vivo administration of soluble MHC class I tetramers. Journal of Immunology (Baltimore, Md.: 1950), 167 (7), 3708-3714. https://doi.org/10.4049/jimmunol.167.7.3708
Majzner, R. G., & Mackall, C. L. (2019). Clinical lessons learned from the first leg of the CAR T cell journey. Nature Medicine, 25 (9), 1341-1355. https://doi.org/10.1038/s41591-019-0564-6
Martinez Marina et al. CAR T Cells for Solid Tumors: New Strategies for Finding, Infiltrating, and Surviving in the Tumor Microenvironment. Frontiers in Immunology, February 2019|Volume 10|Article 128.
Megrelis, L., El Ghoul, E., Moalli, F., Versapuech, M., Cassim, S., Ruef, N., Stein, J. V, Mangeney, M., & Delon, J. (2018). Fam65b Phosphorylation Relieves Tonic RhoA Inhibition During T Cell Migration. Frontiers in Immunology, 9, 2001. https://doi.org/10.3389/fimmu.2018.02001
Metelo A M, Jozwik A, Luong L A, Dominey-Foy D, Graham C, Attwood C, et al. Allogeneic Anti-BCMA CAR T Cells Are Superior to Multiple Myeloma-derived CAR T Cells in Preclinical Studies and May Be Combined with Gamma Secretase Inhibitors. Cancer Res Commun. 2022; 2:158-71.
Nagashima T, Shigematsu N, Maruki R, Urano Y, Tanaka H, Shimaya A, et al. Discovery of novel forkhead box O1 inhibitors for treating type 2 diabetes: improvement of fasting glycemia in diabetic db/db mice. Mol Pharmacol. United States; 2010; 78:961-70.
Neelapu, S. S., Locke, F. L., Bartlett, N. L., Lekakis, L. J., Miklos, D. B., Jacobson, C. A., Braunschweig, I., Oluwole, O. O., Siddiqi, T., Lin, Y., Timmerman, J. M., Stiff, P. J., Friedberg, J. W., Flinn, I. W., Goy, A., Hill, B. T., Smith, M. R., Deol, A., Farooq, U., . . . . Go, W. Y. (2017). Axicabtagene Ciloleucel CAR T-Cell Therapy in Refractory Large B-Cell Lymphoma. The New England Journal of Medicine, 377 (26), 2531-2544. https://doi.org/10.1056/NEJMoa1707447
Novis, C. L., Archin, N. M., Buzon, M. J., Verdin, E., Round, J. L., Lichterfeld, M., Margolis, D. M., Planelles, V., & Bosque, A. (2013). Reactivation of latent HIV-1 in central memory CD4+ T cells through TLR-1/2 stimulation. Retrovirology, 10, 119. https://doi.org/10.1186/1742-4690-10-119.
Okuma Atsushi. Generation of CAR-T Cells by Lentiviral Transduction. Methods Mol Biol. 2021; 2312:3-14.
Rougerie, P., Largeteau, Q., Megrelis, L., Carrette, F., Lejeune, T., Toffali, L., Rossi, B., Zeghouf, M., Cherfils, J., Constantin, G., Laudanna, C., Bismuth, G., Mangeney, M., & Delon, J. (2013). Fam65b is a new transcriptional target of FOXO1 that regulates RhoA signaling for T lymphocyte migration. Journal of Immunology (Baltimore, Md.: 1950), 190 (2), 748-755. https://doi.org/10.4049/jimmunol.1201174
Roux, A., Leroy, H., De Muylder, B., Bracq, L., Oussous, S., Dusanter-Fourt, I., Chougui, G., Tacine, R., Randriamampita, C., Desjardins, D., Le Grand, R., Bouillaud, F., Benichou, S., Margottin-Goguet, F., Cheynier, R., Bismuth, G., & Mangeney, M. (2019). FOXO1 transcription factor plays a key role in T cell-HIV-1 interaction. PLOS Pathogens, 15(5), e1007669. https://doi.org/10.1371/journal.ppat.1007669
Schuster, S. J., Bishop, M. R., Tam, C. S., Waller, E. K., Borchmann, P., McGuirk, J. P., Jäger, U., Jaglowski, S., Andreadis, C., Westin, J. R., Fleury, I., Bachanova, V., Foley, S. R., Ho, P. J., Mielke, S., Magenau, J. M., Holte, H., Pantano, S., Pacaud, L. B., . . . Maziarz, R. T. (2019). Tisagenlecleucel in Adult Relapsed or Refractory Diffuse Large B-Cell Lymphoma. The New England Journal of Medicine, 380 (1), 45-56. https://doi.org/10.1056/NEJMoa1804980
Sun Yingjia et al. Integrated Discovery of FOXO1-DNA Stabilizers from Marine Natural Products to Restore Chemosensitivity to anti-EGFR-based Therapy for Metastatic Lung Cancer. Mol. BioSyst., 2016, DOI: 10.1039/C6MB00678G.
Tanchot, C., Guillaume, S., Delon, J., Bourgeois, C., Franzke, A., Sarukhan, A., Trautmann, A., & Rocha, B. (1998). Modifications of CD8+ T cell function during in vivo memory or tolerance induction. Immunity, 8 (5), 581-590. https://doi.org/10.1016/s1074-7613 (00) 80563-4
Urak, R., Walter, M., Lim, L., Wong, C. W., Budde, L. E., Thomas, S., Forman, S. J., & Wang, X. (2017). Ex vivo Akt inhibition promotes the generation of potent CD19CAR T cells for adoptive immunotherapy. Journal for Immunotherapy of Cancer, 5, 26. https://doi.org/10.1186/s40425-017-0227-4
van der Waart, A. B., van de Weem, N. M. P., Maas, F., Kramer, C. S. M., Kester, M. G. D., Falkenburg, J. H. F., Schaap, N., Jansen, J. H., van der Voort, R., Gattinoni, L., Hobo, W., & Dolstra, H. (2014). Inhibition of Akt signaling promotes the generation of superior tumor-reactive T cells for adoptive immunotherapy. Blood, 124 (23), 3490-3500. https://doi.org/10.1182/blood-2014-05-578583
Zwang N A, Turka L A. Homeostatic expansion as a barrier to lymphocyte depletion strategies. Curr Opin Organ Transplant. 2014; 19:357-62.

1. FOXO1 inhibited CAR-T cells presenting:
an anti-tumor activity which is increased compared to CAR-T cells non-treated by FOXO1 inhibitor; and
a cell expansion which is increased compared to CAR-T cells non-treated by FOXO1 inhibitor.

2. The FOXO1 inhibited CAR-T cells according to claim 1, said FOXO1 inhibited CAR-T cells further lacking the exhausted characteristics of CAR-T cells non-treated by FOXO1 inhibitor.

3. The FOXO1 inhibited CAR-T cells according to claim 1, said FOXO1 inhibitor being the AS1842856.

4. The FOXO1 inhibited CAR-T cells according to claim 1, wherein the anti-tumor activity of 2×10⁵ FOXO1 inhibited CAR-T cells leads to a tumor regression in vivo in a subcutaneous xenograft model of pancreatic Capan-2 cell line expressing luciferase tumor cells, injected in immunodeficient NSG mice, with tumor regression being quantitatively assessed by bioluminescence imaging, whereas 2×10⁵ CAR-T cells non-treated by FOXO1 inhibitor leads to non-controlled tumor in the same subcutaneous xenograft model,
and/or
the anti-tumor activity of 2×10⁵ FOXO1 inhibited CAR-T cells leads after 28 days to the inhibition of at least twice of a tumor progression in vivo in an orthotopic model of A549 cell line expressing luciferase tumor cells, injected in immunodeficient NSG mice, with tumor progression being quantitatively assessed by bioluminescence imaging, as compared to 2×10⁵ CAR-T cells non-treated by FOXO1 inhibitor in the same orthotopic model.

5. The FOXO1 inhibited CAR-T cells according to claim 1, said increased cell expansion being associated with a cell expansion at least 3-fold compared to CAR-T cells non-treated by FOXO1 inhibitor after 10 days of in vitro stimulation.

6. The FOXO1 inhibited CAR-T cells according to claim 1, said FOXO1 inhibited CAR-T cells further lacking the exhausted characteristics of CAR-T cells non-treated by FOXO1 inhibitor,
said lack of exhausted characteristics of CAR-T cells non-treated by FOXO1 inhibitor being associated with a reduction in the percentage of positive said FOXO1 inhibited CAR-T cells of at least 2-fold for Tim3 marker and a reduction in the percentage of positive said FOXO1 inhibited CAR-T cells of at least 5-fold for LAG3 marker.

7. An Ex vivo process for obtaining FOXO1 inhibited CAR-T cells comprising the following steps:
(i) cultivating T cells obtained from a subject with a FOXO1 inhibitor during a time of 2 to 10 days to obtain FOXO1 inhibited T cells.
(ii) transducing said FOXO1 inhibited T cells into FOXO1 inhibited CAR-T cells.

8. The Ex vivo processes for obtaining FOXO1 inhibited CAR-T cells according to claim 7, wherein the FOXO1 inhibitor is the AS1842856.

9. The Ex vivo process for obtaining improved FOXO1 inhibited CAR-T cells according to claim 7, comprising the following steps:
(i) providing T cells from a subject;
(ii) cultivating the T cells with a FOXO1 inhibitor during a time of 2 to 10 days to obtain FOXO1 inhibited T cells.
(iii) transducing said FOXO1 inhibited T cells into FOXO1 inhibited CAR-T cells.

10. The Ex vivo process for obtaining improved FOXO1 inhibited CAR-T cells according to claim 7, wherein the method further comprises a step of addition of IL-7 and/or IL-15 after the use of the inhibitor of FOXO1 and before transducing the FOXO1 inhibited T cells into FOXO1 inhibited CAR-T cells.

11. A method to treat a cancer or an infectious disease in a subject in need thereof comprising administering to the subject a therapeutically effective amount of a FOXO1 inhibited CAR-T cells presenting:
an anti-tumor activity which is increased compared to CAR-T cells non-treated by FOXO1 inhibitor; and
a cell expansion which is increased compared to CAR-T cells non-treated by FOXO1 inhibitor.

12. The method to treat a cancer or an infectious disease according to claim 11, said FOXO1 inhibited CAR-T cells further lacking the exhausted characteristics of CAR-T cells non-treated by FOXO1 inhibitor.

13. The method to treat a cancer or an infectious disease according to claim 11, wherein the anti-tumor activity of 2×10⁵ FOXO1 inhibited CAR-T cells leads to a tumor regression in vivo in a subcutaneous xenograft model of pancreatic Capan-2 cell line expressing luciferase tumor cells, injected in immunodeficient NSG mice, with tumor regression being quantitatively assessed by bioluminescence imaging, whereas 2×10⁵ CAR-T cells non-treated by FOXO1 inhibitor leads to non-controlled tumor in the same subcutaneous xenograft model,
and/or
the anti-tumor activity of 2×10⁵ FOXO1 inhibited CAR-T cells leads after 28 days to the inhibition of at least twice of a tumor progression in vivo in an orthotopic model of A549 cell line expressing luciferase tumor cells, injected in immunodeficient NSG mice, with tumor progression being quantitatively assessed by bioluminescence imaging, as compared to 2×10⁵ CAR-T cells non-treated by FOXO1 inhibitor in the same orthotopic model.

14. The method to treat a cancer or an infectious disease according to claim 11, said increased cell expansion being associated with a cell expansion at least 3-fold compared to CAR-T cells non-treated by FOXO1 inhibitor after 10 days of in vitro stimulation.

15. The method to treat a cancer or an infectious disease according to claim 11, said FOXO1 inhibited CAR-T cells further lacking the exhausted characteristics of CAR-T cells non-treated by FOXO1 inhibitor,
said lack of exhausted characteristics of CAR-T cells non-treated by FOXO1 inhibitor being associated with a reduction in the percentage of positive said FOXO1 inhibited CAR-T cells of at least 2-fold for Tim3 marker and a reduction in the percentage of positive said FOXO1 inhibited CAR-T cells of at least 5-fold for LAG3 marker.

16. The method to treat a cancer or an infectious disease according to claim 11, said cancer being a liquid or a solid cancer.

17. The method to treat a cancer or an infectious disease according to claim 11, said cancer being selected from the group consisting in: adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer (e.g. uterine leiomyosarcoma).

18. The method to treat a cancer or an infectious disease according to claim 11, said infectious diseases being due to a pathogen such as a virus, bacterium, protozoan, prion, viroid, or fungus.

19. The method to treat a cancer or an infectious disease according to claim 11, said infectious diseases being due to a bacterium selected from the group consisting of: Streptococcus pneumoniae; Staphylococcus aureus; Haemophilus influenza, Myoplasma species, Moraxella catarrhalis, Escherichia, Enterobacter, Erwinia, Klebsiella, Proteus, Salmonella, Typhimurium, Serratia, Shigella, Bacilli, Pseudomonas, Campylobacter, Mycobacterium tuberculosis and Streptomyce or said infectious diseases being due to a fungus selected from the group consisting of:
Aspergillus, Candida albicans and Cryptococcus neoformans,
or said infectious diseases being due to a respiratory virus selected from the group consisting of: Influenza virus, adenovirus, metapneumovirus, cytomegalovirus, parainfluenza virus, the human rhinovirus (HRV), the Human respiratory syncytial virus (HRSV) and a coronavirus.

20. The method to treat a cancer or an infectious disease according to claim 11, said therapeutically effective amount of a FOXO1 inhibited CAR-T cells being from 10⁴ to 10⁹ cells per kg.
